(19) United States
(12) Patent Application Publication
(10) Pub. No.: US 2021/0204574 A1
(43) Pub. Date: Jul. 8, 2021

(54) Consumable Product Comprising Malted Wheat

(71) Applicant: Lantmannen Functional Foods AB, Stockholm (SE)

(72) Inventors: Ivar Lönnroth, Mölndal (SE); Lin Shi, Xi'an (CN); Rikard Landberg, Göteborg (SE)

(73) Assignee: Lantmannen Functional Foods AB, Stockholm (SE)

(21) Appl. No.: 17/193,141

(22) Filed: Mar. 5, 2021

Related U.S. Application Data
(63) Continuation of application No. PCT/EP2019/076399, filed on Sep. 30, 2019.

(30) Foreign Application Priority Data
Sep. 28, 2018 (SE) 1851169-1

(51) Int. Cl. A23L 7/25; A23L 29/00; A23L 33/00; A61P 1/04; A23K 10/30; A23K 20/121; A61K 31/7042; A61K 31/192; A61K 31/09; A61K 31/538; A23K 20/111

(52) U.S. Cl. CPC A23L 7/25; A23L 29/035; A23L 33/30; A61P 1/04; A23K 10/30; A23L 29/045; A61K 31/7042; A61K 31/192; A61K 31/09; A61K 31/538; A23K 20/111; A23K 20/121

(57) ABSTRACT

The disclosure relates to a consumable product comprising malted wheat and/or a leachate of malted wheat, wherein said consumable product induces endogenous production of antisecretory factor (AF) protein and/or fragments thereof in a subject after consumption. The malted wheat of the consumable product comprises (i) 2-methoxyphenol and/or a derivate thereof, and (ii) 2,4-dihydroxy-7-methoxy-(2)-1,4-benzoxazin-3(4H)-one derivative, wherein (a) the concentration of (i) is higher and/or substantially the same as compared to the corresponding non-malted wheat, and (b) the concentration of (ii) is higher as compared to the corresponding non-malted wheat.

The present disclosure further relates to a consumable product produced in accordance with the herein described process, and to use of the consumable product as food or feed for humans and/or animals as well as for medical uses.

This application is a continuation of PCT Patent Application No. PCT/EP2019/076399, filed on Sep. 30, 2019, which claims priority from Swedish Patent Application No. 1851169-1, filed on Sep. 28, 2018. The contents of these priority applications are incorporated herein by reference in their entirety.

### TECHNICAL FIELD

The present disclosure relates to a consumable product comprising malted wheat, wherein said consumable product induces endogenous production of antisecretory factor (AF) protein and/or fragments thereof in a subject after consumption. The malted wheat of the consumable product comprises (i) a 2-methoxyphenol and (ii) a 2,4-dihydroxy-7-methoxy-(2H)-1,4-benzoxazin-3(4H)-one-derivative, herein referred to as a DIMBOA-derivative, selected from the group consisting of DIMBOA_hex and DIMBOA_hex_hex, as described herein, wherein (a) the concentration of (i) is higher and/or substantially the same as compared to in the corresponding non-malted wheat, and (b) the concentration of (ii) is higher as compared to in the corresponding non-malted wheat.

In one embodiment, the present disclosure relates to a consumable product comprising leachate of malted wheat, wherein said leachate of malted wheat comprises 2,4-dihydroxy-7-methoxy-(2H)-1,4-benzoxazin-3(4H)-one-hex at a concentration of at least 20.000 ng/ml and 2,4-dihydroxy-7-methoxy-(2H)-1,4-benzoxazin-3(4H)-one-hex-hex at a concentration of at least 700 ng/ml.

In another embodiment, the present disclosure relates to a consumable product comprising malted wheat, wherein said malted wheat comprises 2,4-dihydroxy-7-methoxy-(2H)-1,4-benzoxazin-3(4H)-one-hex at a concentration of at least 60 ng/mg and 2,4-dihydroxy-7-methoxy-(2H)-1,4-benzoxazin-3(4H)-one-hex-hex at a concentration of at least 2 ng/mg.

The present disclosure further relates to a consumable product produced in accordance with the herein described process, and to a use of the consumable product as food or feed for humans and/or animals.

### BACKGROUND

### Antisecretory Factor (AF) Protein

The antisecretory factor (AF) is a class of proteins that occurs naturally in the body. Antisecretory factor (AF) protein is a 41 kDa protein that was originally described to provide protection against diarrhea diseases and intestinal inflammation (for a review, see Lange and Lönnroth, 2001). The antisecretory factor (AF) protein has long since been sequenced and its cDNA cloned (see SEQ ID NO: 1). The antisecretory activity seems to be mainly exerted by a peptide located between the amino acid positions 35 and 50 on the antisecretory factor (AF) protein sequence which comprises at least 4-16, such as 4, 6, 7, 8 or 16 amino acids of the consensus sequence. The biological effect of AF is exerted by any peptide or polypeptide comprising at least 6 amino acids as shown in SEQ ID NO: 2 (AF-6), of said consensus sequence, or a modification thereof not altering the function of the polypeptide and/or peptide, such as by a peptide as shown in SEQ ID NO: 3 (AF-16), or in SEQ ID NO: 4 (AF-8).

It has been shown that the antisecretory factor (AF) protein is to some extent homologous with the protein S5a, and Rpn10, which constitutes a subunit of a constituent prevailing in all cells, the 26 S proteasome, more specifically in the 19 S/PA 700 cap. In the present disclosure, antisecretory factor (AF) proteins are defined as a class of homologue proteins having the same functional properties. Antisecretory factor (AF) protein is also highly similar to angiocidin, another protein isoform known to bind to thrombospondin-1 and associated with cancer progression.

Immunochemical and immunohistochemical investigations have revealed that the antisecretory factor (AF) protein is present and may also be synthesized by most tissues and organs in a body.

Synthetic peptides, comprising the antidiarrheal sequence, have prior been characterized (see WO 97/08202; WO 05/030246; WO 2007/126364; WO 2018/015379).

Antisecretory factor (AF) proteins and peptides (ASPs) have previously been disclosed to normalize pathological fluid transport and/or inflammatory reactions, such as in the intestine and in the central nervous system after challenge with the cholera toxin (WO 97/08202). WO97/08202 discloses structures of certain antisecretory proteins, and their active parts are characterized. A synthetic ASP prepared by recombinant genetic engineering or by solid phase technology and having definite structures has been shown to have a general controlling influence on the body fluid flow over living cell membranes.

Food and feed with the capacity to either induce endogenous synthesis of AF or uptake of added AF have therefore been suggested to be useful for the treatment of edema, diarrhea, dehydration and inflammation in WO 97/08202. WO 98/21978 discloses the use of products having enzymatic activity for the production of a food that induces the formation of antisecretory factor (AF) proteins after consumption. WO 00/038535 further discloses food products enriched and/or naturally rich in native antisecretory factor (AF) proteins as such.

Antisecretory factor (AF) proteins and fragments thereof have also been shown to improve the repair of nervous tissue, and proliferation, apoptosis, differentiation, and/or migration of stem and progenitor cells and cells derived thereof in the treatment of conditions associated with loss and/or gain of cells (WO 05/030246) and to be equally effective in the treatment and/or prevention of intraocular hypertension (WO 07/126364), as for the treatment and/or prevention of compartment syndrome (WO 07/126363).

From the Swedish Patent SE 9000028-2 (publication No. 466,331) it is known that the formation of an antisecretory factor (AF) or an antisecretory factor (AF) protein (in SE 9000028-2 named ASP: also named FIL) can be stimulated by adding, to the animals' feed, certain sugars, amino acids and amides. The kinds and amounts of these substances to be used for the formation of an effective amount of ASP is determined by a method disclosed in the patent. Briefly, this method involves measurement of a standardized secretion response in the small intestine of rat. From the patent, it is evident that the induced ASPs formed direct the secretion of body fluid into the intestine. In said patent, the content or amount of natural antisecretory proteins is defined by its effect on the fluid secretion into the small intestine of laboratory rats having been challenged with cholera toxin (RTT-test). One ASP Unit (FIL Unit) corresponds to a 50% reduction of the fluid flow in the rat's intestine compared to a control without ASP. The antisecretory proteins are active in extremely small amounts and, therefore, it is often easier to determine them by their effect than by their mass.

From WO 98/21978 it is known that the formation of ASP can be induced in the body by consumption of a certain kind of food having enzymatic activity. The effect of the induction and, owing to that, the formation of ASP varies according to the individual and its symptoms and takes place with a strength and induction period unpredictable so far. However, they can be measured afterwards, and necessary corrections can be made with the guidance of said measurements. It is mentioned that the products may be malted cereals.

### Benzoxazinoids

WO 2009/115093 discloses use of grains or disintegrated grains of benzoxazinoids-containing cereals for the manufacturing of a food product with health-improving effects. It is disclosed that hydrothermal pretreatment of the grains resulted in an increased content of benzoxazinoids.

It is an object of the present disclosure to provide a consumable product such as a food, feed and/or food- or feed-supplement comprising malted wheat and/or a leachate of said malted wheat that after malting comprises a sufficiently high enough amount of compounds that stimulate and/or induce endogenous production of antisecretory factor (AF) protein, peptides and/or fragments thereof in a subject, such as a human or an animal after consumption. Further, it is an object of the present disclosure to overcome or at least alleviate some of the disadvantages of known processes for producing food products comprising malted wheat and/or a leachate of said malted wheat that after malting comprise compounds such as phenolic acids and/or benzoxazinoids with health improving effects.

### Definitions and Abbreviations

Proteins are biological macromolecules constituted by amino acid residues linked together by peptide bonds. Proteins, as linear polymers of amino acids, are also called polypeptides. Typically, proteins have 50-800 amino acid residues and hence have molecular weights in the range of from about 6,000 to about several hundred thousand Dalton or more. Small proteins are called peptides, polypeptides, or oligopeptides. The terms “protein”, “polypeptide”, “oligopeptide” and “peptide” may be used interchangeably in the present context. Peptides can have very few amino acid residues, such as between 2-50 amino acid residues (aa).

The term “antisecretory” refers in the present context to inhibiting or decreasing secretion and/or fluid transfer. Hence, the term “antisecretory factor (AF) protein” refers to a class of proteins capable of inhibiting or decreasing or otherwise modulating fluid transfer as well as secretion in a body.

In the present context, the terms an “antisecretory factor protein”, “antisecretory factor (AF) protein”, “AF-protein”, AF, or a homologue, derivative or fragment thereof, may be used interchangeably with the term “antisecretory factors” or “antisecretory factor proteins” as defined in WO 97/08202, and refer to an antisecretory factor (AF) protein or a peptide or a homologue, derivative and/or fragment thereof having antisecretory and/or equivalent functional and/or analogue activity, or to a modification thereof not altering the function of the polypeptide. Hence, it is to be understood that an “antisecretory factor”, “antisecretory factor protein”, “antisecretory peptide”, “antisecretory fragment”, or an “antisecretory factor (AF) protein” in the present context, also can refer to a derivative, homologue or fragment thereof. These terms may all be used interchangeably in the context of the present disclosure. Furthermore, in the present context, the term “antisecretory factor” may be abbreviated “AF”. Antisecretory factor (AF) protein in the present context also refers to a protein with antisecretory properties as previously defined in WO97/08202 and WO 00/38535. Antisecretory factors have also been disclosed e.g. in WO 05/030246.

The term “ASP” is in the present context used for “antisecretory protein” i.e. natural antisecretory factor (AF) protein.

In the present context “AF activity” is measured as elevation of AF-Units in the blood after consumption of the consumable product of the present invention by inducing more than 0.5, such as at least 0.6, 0.7, 0.8, 0.9, 1, 1.5 or 2 AF-Units/ml blood in a human or an animal. Increased AF activity is defined by its effect on the fluid secretion into the small intestine of laboratory rats having been challenged with cholera toxin (RTT-test/ligated loop assay). One ASP/AF-Unit (FIL-Unit) corresponds to a 50% reduction of the fluid flow in the rat's intestine compared to a control without ASP, i.e. corresponding to approximately to 1.5 nM AF protein per liter plasma (1.5 nM/L).

AF activity can also be measured by the use of a kit, an assay and/or a method as described in WO 2015/181324 (Antisecretory Factor Complex Assay) for verifying effectiveness of a consumable product according to the present invention as compliance of human and/or animals to the same consumable product after consumption.

By “functional food product” is meant, in the present context, a food product having a salubrious function, i.e. having a beneficial effect on the health of a human or an animal.

In the present context, the expression “pathologically high levels of body fluid discharge” means levels of body fluid discharge such as from intracellular fluid and/or extracellular fluid, the latter being selected from the group consisting of intravascular fluid, interstitial fluid, lymphatic fluid and transcellular fluid, that deviate from what is considered normal and/or healthy in a human and/or animal. Specifically, the levels of body fluid discharge may be such that it may be considered by a health care professional such as a nurse or a physician appropriate to treat the patient. In the present context, the term “pathological” is used to in general describe an abnormal anatomical or physiological condition. The term “disease pathology” in general encompasses the causes, processes and changes in body organs and tissues that occur with human illness. Many of the most common pathological diseases are causes of death and disability.

AF: antisecretory factor, antisecretory factor (AF) protein
Full-length AF protein (as shown in SEQ ID NO: 1)
AF-6: a hexapeptide CHSKTR (as shown in SEQ ID NO: 2);
AF-16: a peptide composed of the amino acids VCHSKTRSNPENNVGL (as shown in SEQ ID NO: 3);
AF-8: a septa peptide VCHSKTR (as shown in SEQ ID NO: 4);
Octa peptide IVCHSKTR (as shown in SEQ ID NO: 5);
RTT: Method for measuring a standardized secretion response in rat small intestine, as published in SE 9000028-2 (publication number 466331) for measuring content of AF (ASP) in blood.
glc: glucose
hex: hexose
g: gram(s)
ml: milliliter(s)
μL: microliter(s)

### min.: Minute(s)

vol: volume

### UPLC: Ultra Performance Liquid Chromatography

### V: Volt(s)

### GHz: GigaHertz

LC: Liquid chromatography

### Q-TOF: Quadropole Time of Flight MS (High Resolution Mass Spectroscopy)

### RP: Reverse Phase

### MS: Mass Spectroscopy

rpm: revolutions per minute
ppm: part per million
obiwarp: Ordered Bijective Interpolated Warping
mzML: mz (mass to charge ratio)

### SUMMARY

The present disclosure relates to a consumable product comprising malted wheat and/or a leachate of malted wheat, wherein said malted wheat of the consumable product comprises (i) a 2-methoxyphenol and (ii) a 2,4-dihydroxy-7-methoxy-(2H)-1,4-benzoxazin-3(4H)-one derivative, herein referred to as a DIMBOA-derivative, selected from the group consisting of DIMBOA_hex and DIMBOA_hex_hex, as described herein, wherein (a) the concentration of (i) is higher and/or substantially the same as compared to in the corresponding non-malted wheat, and (b) the concentration of (ii) is higher as compared to in the corresponding non-malted wheat.

In particular, the present disclosure in one aspect relates to a consumable product comprising leachate of malted wheat, wherein said leachate of malted wheat comprises (i) a 2-methoxyphenol and (ii) a 2,4-dihydroxy-7-methoxy-(2H)-1,4-benzoxazin-3(4H)-one derivative, selected from the group consisting of DIMBOA_hex and DIMBOA_hex_hex, as described herein, wherein (a) the concentration of (i) is higher and/or substantially the same as compared to in the corresponding non-malted wheat, and (b) the concentration of 2,4-dihydroxy-7-methoxy-(2H)-1,4-benzoxazin-3(4H)-one-hex is at least 20.000 ng/ml and the concentration of 2,4-dihydroxy-7-methoxy-(2H)-1,4-benzoxazin-3(4H)-one-hex-hex is at least 700 ng/ml.

In another aspect, the present disclosure relates to a consumable product comprising malted wheat, wherein said malted wheat comprises (i) a 2-methoxyphenol and (ii) a 2,4-dihydroxy-7-methoxy-(2H)-1,4-benzoxazin-3(4H)-one derivative, selected from the group consisting of DIMBOA_hex and DIMBOA_hex_hex, as described herein, wherein (a) the concentration of (i) is higher and/or substantially the same as compared to in the corresponding non-malted wheat, and (b) the concentration of 2,4-dihydroxy-7-methoxy-(2H)-1,4-benzoxazin-3(4H)-one-hex is at least 60 ng/mg and the concentration of 2,4-dihydroxy-7-methoxy-(2H)-1,4-benzoxazin-3(4H)-one-hex-hex is at least 2 ng/mg.

The consumable product disclosed herein induces endogenous production of antisecretory factor (AF) protein and/or fragments thereof in a subject after consumption. The extent of the induction of said endogenous production of the antisecretory factor (AF) protein and/or fragments thereof may be adjusted by providing an appropriate amount of the consumable product to a subject in need thereof.

Consequently, the consumable product of the present invention may be used in the treatment, prevention and/or prophylaxis of an abnormal physiological condition characterized by and/or associated with elevated and/or pathologically high levels of body fluid discharge. Further, the consumable product of the present invention may be used in a treatment and/or prevention of a condition responsive to increased levels of antisecretory factor protein and/or antisecretory protein fragments in the blood of a patient. For instance, the consumable product may be used to treat diarrhoea, oedema and/or conditions involving inflammation in a subject such as a human and/or an animal. In a further example, the condition to be treated with the consumable product described herein may be selected from the group consisting of diarrhoea, inflammatory disease, oedema, autoimmune disease, cancer, tumour, leukaemia, diabetes, diabetes mellitus, glioblastoma, traumatic brain injury, intraocular hypertension, glaucoma, lipid raft dysfunction, compartment syndrome, Alzheimer's disease, Parkinson's disease, encephalitis, and Meniere's disease.

In one example, the compound (i) may comprise at least one 2-methoxyphenol derivative, selected from the group consisting of ferulic acid, sinapinic acid and vanillic acid.

In one example, the compound (ii) is a hexose derivative of 2,4-dihydroxy-(2H)-1,4-benzoxazin-3(4H)-one.

The malted wheat and/or leachate thereof comprised in the consumable product may comprise a further compound (iii) such as 2,4-dihydroxy-(2H)-1,4-benzoxazin-3(4H)-one, a hexose derivative of 2,4-dihydroxy-(2H)-1,4-benzoxazin-3(4H)-one and/or benzoxazoline-2-one, wherein the concentration of (iii) is higher as compared to in the corresponding non-malted wheat.

Further, the consumable product may comprise malted wheat and/or leachate thereof in which the concentration of any one of the compounds (ii) is higher than that of any one of the compounds (iii).

The malted wheat of the consumable product may be obtained from a process comprising the steps of:

a. malting wheat at a temperature from about 5° C. to about 20° C., and subsequent
b. drying said wheat at no more than 80° C.

More specifically, the malted wheat of the consumable product may be obtained from a process comprising the steps of:

a. wet steeping of wheat at a temperature from about 5° C. to about 20° C.,
b. optionally drying of said wheat,
c. growing of said wheat at a temperature from about 5° C. to about 20° C.,
d. optionally repeating any one of said steps a-c, and subsequent
e. drying of said wheat at no more than 80° C.

In the above-mentioned process, step a. and/or step b. and/or step c. may independently take place at a temperature of about 8° C., or from about 13° C. to about 15° C.

The malted wheat and/or leachate thereof of the consumable product may be provided from a wheat variety selected from the group consisting of Kosack, Festival, Stava and any combination thereof. For instance, the wheat variety may be Festival and/or Stava.

The malted wheat and/or leachate thereof of the consumable product may be provided from a wheat variety selected to be genetically closely related to any one of the wheat varieties of the group consisting of Kosack, Festival, Stava. In particular, the malted wheat and/or leachate thereof of the consumable product may be provided from a wheat variety selected to after malting with a malting process according to the one of the present disclosure comprise (i) a 2-methoxyphenol and (ii) a 2,4-dihydroxy-7-methoxy-(2H)-1,4-benzoxazin-3(4H)-one derivative, herein referred to as a DIMBOA-derivative, selected from the group consisting of DIMBOA_hex and DIMBOA_hex_hex, as described herein, wherein (a) the concentration of (i) is higher and/or substantially the same as compared to in the corresponding non-malted wheat, and (b) the concentration of (ii) is higher as compared to in the corresponding non-malted wheat, in particular again wherein (b) the concentration of 2,4-dihydroxy-7-methoxy-(2H)-1,4-benzoxazin-3(4H)-one-hex is at least 20.000 ng/ml and the concentration of 2,4-dihydroxy-7-methoxy-(2H)-1,4-benzoxazin-3(4H)-one-hex-hex is at least 700 ng/ml, or wherein (b) the concentration of 2,4-dihydroxy-7-methoxy-(2H)-1,4-benzoxazin-3(4H)-one-hex is at least 60 ng/mg and the concentration of 2,4-dihydroxy-7-methoxy-(2H)-1,4-benzoxazin-3(4H)-one-hex-hex is at least 2 ng/mg.

The consumable product may be provided as a food, feed, food supplement, feed supplement and/or a nutraceutical. The food may be food for human consumption such as but not limited to a functional food. The feed may be feed for animal consumption such as feed for poultry and/or livestock animals. The consumable product may be provided as a dry or semi-dry food and/or feed substance, or as a liquid. In one embodiment, the food and/or feed is provided as an infusion. Further, the consumable product may be a pharmaceutical product such as a medicament.

### BRIEF DESCRIPTION OF THE DRAWINGS

FIG. 1a shows the chemical structure of DIBOA, DIBOA_hex, DIBOA_dihexose, DIMBOA, DIMBOA_glc, DIMBOA_hex_and DIMBOA_hex_hex.

FIG. 1b shows the chemical structure of DIMBOA_hex.

FIG. 1c shows the chemical structure of DIMBOA_hex_hex.

FIG. 2a shows the chemical structure of 2-methoxyphenol (guiacol).

FIG. 2b shows the chemical structure of ferulic acid.

FIG. 2c shows the chemical structure of vanillic acid.

FIG. 2d shows the chemical structure of sinapic acid

FIG. 3a shows the correlation between the concentration of DIMBOA and antisecretory factor activity.

FIG. 3b shows that the concentration of methyl DIMBOA increases when the wheat is subjected to the malting process as described herein as compared to the corresponding non-malted wheat.

FIG. 4a shows the correlation between the concentration of DIMBOA_hex_hex and antisecretory factor activity.

FIG. 4b shows that the concentration of methyl DIMBOA_hex_hex increases when the wheat is subjected to the malting process as described herein as compared to the corresponding non-malted wheat.

FIG. 5a shows the correlation between the concentration of DIMBOA_glc and antisecretory factor activity.

FIG. 5b shows that the concentration of methyl DIMBOA_glc increases when the wheat is subjected to the malting process as described herein as compared to the corresponding non-malted wheat.

FIG. 6a shows the correlation between the concentration of BOA and antisecretory factor activity.

FIG. 6b shows that the concentration of BOA increases when the wheat is subjected to the malting process as described herein as compared to the corresponding non-malted wheat.

FIG. 7a shows the correlation between the concentration of DIBOA and antisecretory factor activity.

FIG. 7b shows that the concentration of DIBOA increases when the wheat is subjected to the malting process as described herein as compared to the corresponding non-malted wheat.

FIG. 8a shows the correlation between the concentration of DIBOA_hex and antisecretory factor activity.

FIG. 8b shows that the concentration of DIBOA_hex increases when the wheat is subjected to the malting process as described herein as compared to the corresponding non-malted wheat.

FIG. 9a shows the correlation between the concentration of DIBOA_dihexose and antisecretory factor activity.

FIG. 9b shows that the concentration of DIBOA_hex_hex increases when the wheat is subjected to the malting process as described herein as compared to the corresponding non-malted wheat.

FIG. 10a shows that the AF activity in blood was significantly increased in the animals receiving guaiacol and ferulic acid, and the leachate from Kosack wheat malt also showed activity. Sprague-Dawley rats were given 5 μM ferulic acid (n=10), 5 μM guaiacol (n=10) or leachate from wheat malt (n=5) in drinking water for 14 days or just water as control (n=9). The content of AF in blood plasma was measured with ELISA using a monoclonal antibody against AF as catching antibody and a polyclonal antibody against complement C3 as detecting antibody. The AF value is given as reversed titre. The proteasome/C3 complex was significantly higher in the ferulic acid, guaiacol, and malt-treated rats compared to the controls (p<0.05, p<0.01, and p<0.01 respectively).

FIG. 10b shows that the AF activity in blood was significantly increased in the animals receiving catechin and sinapic acid, and the leachate from Kosack wheat malt also showed activity (positive control). Induction of AF activity in rat blood. Rats, 5 animals/group, were given 10 μm catechin, ferulic acid or SPC in the drinking water for 14 days. The graph shows the AF activity in blood measured with ELISA. Both catechin (p<0.001), sinapic acid (p<0.01) or SPC (p<0.01) gave a significant increase in compleasome concentration.

FIG. 11 Sequence listing

FIG. 12a and FIG. 12b show the activity of AF in blood plasma of rats drinking leachate of malt of different origin; the Festival and Stava leachate showed the highest activity. Sprague-Dawley rats were given leachate of wheat malt in the drinking water for 14 days or just water as control. The content of AF in blood plasma was measured with ELISA using a monoclonal antibody against AF (FIG. 12a) and a polyclonal antibody against complement C3 (FIG. 12b) (Johansson et al. 2009)). The AF and C3 values are given as absorbance at 405 nm. The AQF value was significantly higher in leachate of Stava and Festival wheat malt compared to control rats given tap water (p<0.01, resp, 5 animals per group). The C3 value was significant higher in Festival and Hallfreda wheat malt.

FIG. 13a shows the correlation between the concentration of salicylic acid and antisecretory factor activity.

FIG. 13b shows that the concentration of salicylic acid increases when the wheat is subjected to the malting process as described herein as compared to the corresponding non-malted wheat.

FIG. 14 a and b show correlations between DIMBOA_hex (a) and DIMBOA_hex_hex (b) measured by targeted analysis and untargeted metabolomics (from previous determinations).

### DETAILED DESCRIPTION

The present disclosure is based on the unexpected finding that a consumable product comprising malted wheat, and/or a leachate thereof induces endogenous production of antisecretory factor (AF) protein and/or fragments thereof in a subject after consumption when said malted wheat comprises a combination of (i) 2-methoxyphenol and/or a derivative thereof and (ii) a 2,4-dihydroxy-7-methoxy-(2H)-1,4-benzoxazin-3(4H)-one derivative, wherein (a) the concentration of (i) is higher and/or substantially the same as compared to the corresponding non-malted wheat, and (b) the concentration of (ii) is higher as compared to the corresponding non-malted wheat.

Surprisingly, it was found that that the combination of (i) 2-methoxyphenol and/or a derivate thereof and (ii) a 2,4-dihydroxy-7-methoxy-(2H)-1,4-benzoxazin-3(4H)-one derivative, in the concentrations described herein increased the Antisecretory Factor (AF) activity in subjects after consumption of the consumable product.

Additionally, the compounds (ii) could correlate positively or negatively with Antisecretory Factor (AF) activity. As used herein, a positive correlation with AF activity means that an increase in concentration of the compound is accompanied by an increase in AF activity. Conversely, a negative correlation with AF activity means that a decrease in concentration of the compound is accompanied with a decrease in AF activity.

Thus, there is provided a consumable product comprising malted wheat and/or a leachate of malted wheat comprising

(i) 2-methoxyphenols in one embodiment selected from the group consisting of ferulic acid, sinapic acid and vanillic acid and
(ii) a 2,4-dihydroxy-7-methoxy-(2H)-1,4-benzoxazin-3(4H)-one derivative selected from the group consisting of DIMBOA_hex and DIMBOA_hex_hex, and any combination of the foregoing compounds, wherein (a) the concentration of (i) is higher and/or substantially the same as compared to the corresponding non-malted wheat, and (b) the concentration of (ii) is higher as compared to the corresponding non-malted wheat, and wherein the consumable product induces endogenous production of antisecretory factor (AF) protein and/or fragments thereof in a subject after consumption.

In particular, the present disclosure relates to a consumable product comprising a leachate of malted wheat, wherein said leachate of malted wheat comprises (i) a 2-methoxyphenol and (ii) a 2,4-dihydroxy-7-methoxy-(2H)-1,4-benzoxazin-3(4H)-one derivative, selected from the group consisting of DIMBOA_hex and DIMBOA_hex_hex, wherein (a) the concentration of (i) is higher and/or substantially the same as compared to in the corresponding non-malted wheat, and (b) the concentration of 2,4-dihydroxy-7-methoxy-(2H)-1,4-benzoxazin-3(4H)-one-hex is at least 20 000 ng/ml and the concentration of 2,4-dihydroxy-7-methoxy-(2H)-1,4-benzoxazin-3(4H)-one-hex-hex is at least 700 ng/ml.

The concentration of DIMBOA-derivatives in leachate of malted wheat is measured as described in example 5 by UHPLC-MS/MS analysis, wherein 100 μL of each sample is extracted with 900 μL of 80% methanol, shaken vigorously for 5 min and incubated at +4° C. for two hours. Thereafter, samples are centrifuged at 4° C. for 10 min at 12.000 g and the supernatant is recovered. The amount of the DIMBOA-derivatives is measured against DIMBOA and DIMBOA_hex standards which are prepared in and diluted with 80% methanol and with calibration curves ranging from 10 ng/ml to 10 000 ng/ml. DIMBOA_hex_hex is calibrated by using the DIMBOA_hex calibration curve.

As can be seen in example 5, the concentration of 2,4-dihydroxy-7-methoxy-(2H)-1,4-benzoxazin-3(4H)-one-hex in the leachate of the malted wheat variants that induce AF effectively in subjects after consumption (herein represented by the variants K, S and F) is at least 20 000 ng/ml, such as at least 28 388, 23 370, 28 174, 25 213, 23 520, 31 161, 40 115, 44.291, or 43 860 ng/ml, Thus, the concentration of 2,4-dihydroxy-7-methoxy-(2H)-1,4-benzoxazin-3(4H)-one-hex in the eachate of the malted wheat variants that induce AF effectively in subjects after consumption is between appr. 20 000-45 000 ng/ml, such as at between 23 370-44 291 ng/ml, such as between 23 520-44 291 ng/ml,

As can further be seen in example 5, the concentration of 2,4-dihydroxy-7-methoxy-(2H)-1,4-benzoxazin-3(4H)-one-hex-hex in the eachate of the malted wheat variants that induce AF effectively in subjects after consumption (herein represented by the variants K, S and F) is at least 700 ng/ml, such as at least 1118, 1169, 1102, 731, 837, 787, 1313, 1373, or 1326 ng/ml. Thus, the concentration of 2,4-dihydroxy-7-methoxy-(2H)-1,4-benzoxazin-3(4H)-one-hex-hex in the leachate of the malted wheat variants that induce AF effectively in subjects after consumption is between appr. 700-1400 ng/ml, such as at between ng/ml, such as between 731-1373 ng/ml, such as between 1102-1373 ng/ml, 100 ml leachate corresponds to approximately 33 g coarse grained malted wheat, as is described in example 1 of the present disclosure.

Thus, the present disclosure further in particular relates to a consumable product comprising malted wheat, wherein said malted wheat of the consumable product comprises (i) a 2-methoxyphenol and (ii) a 2,4-dihydroxy-7-methoxy-(2H)-1,4-benzoxazin-3(4H)-one derivative, selected from the group consisting of DIMBOA_hex and DIMBOA_hex_hex, wherein (a) the concentration of (i) is higher and/or substantially the same as compared to in the corresponding non-malted wheat, and (b) the concentration of 2,4-dihydroxy-7-methoxy-(2H)-1,4-benzoxazin-3(4H)-one-hex is at least 60 ng/mg coarse grained malted wheat, such as at least 60, 70, 80, 90 or 100 ng/mg coarse grained malted wheat and the concentration of 2,4-dihydroxy-7-methoxy-(2H)-1,4-benzoxazin-3(4H)-one-hex-hex is at least 2 ng/mg coarse grained malted wheat, such as at least 2, 3, 4, 5, 6, 7, 8, 9 or 10 ng/mg coarse grained malted wheat.

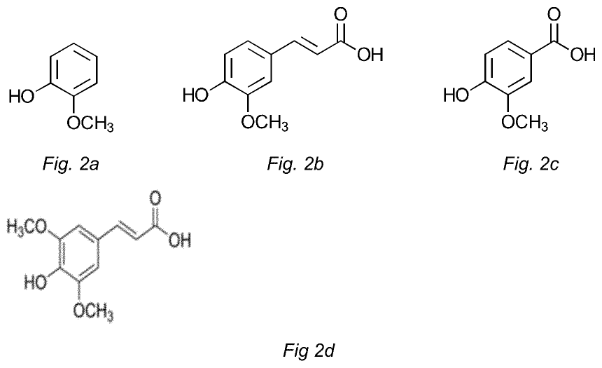

The derivative of 2-methoxyphenol (guaiacol) described herein may be ferulic acid and/or sinapic acid and/or vanillic acid. As shown in FIGS. 2a-2d, ferulic acid and vanillic acid and sinapic acid include a guiacol moiety in their chemical structure.

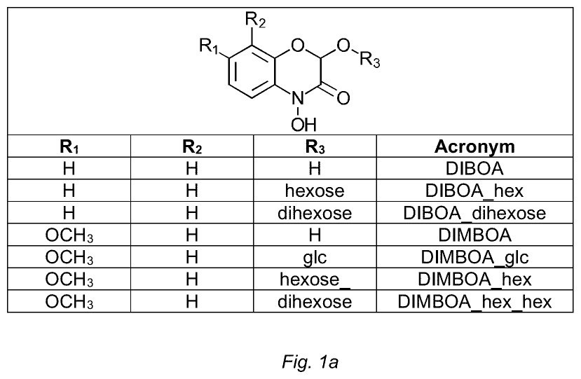

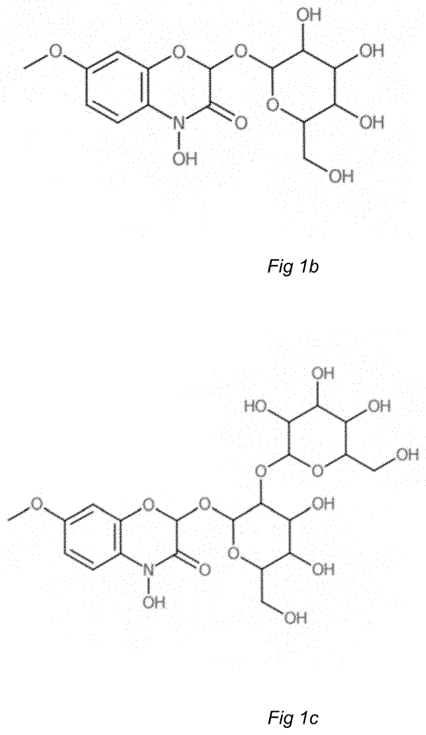

As shown in the figures of the present document, the chemical compounds of (ii) exhibit very different chemical structures. For instance, the chemical compounds of (ii) may be a a hydroxamic acid as depicted in FIG. 1. Yet, they all have a beneficial impact on AF activity in the sense that they increase AF activity with increasing or decreasing concentrations of the compound (ii).

Additionally, or alternatively, the compound (ii) may be selected from the group consisting of: (ii) 2,4-dihydroxy-7-methoxy-(2H)-1,4-benzoxazin-3(4H)-one derivatives. As used herein, 2,4-dihydroxy-7-methoxy-(2H)-1,4-benzoxazin-3(4H)-one may be called DIMBOA. Further, as used herein a derivative of DIMBOA may be a glc or hexose derivative as shown in FIG. 1.

In particular, the compound (ii) may be selected from the group consisting of 2,4-dihydroxy-7-methoxy-(2H)-1,4-benzoxazin-3(4H)-one-hex and 2,4-dihydroxy-7-methoxy-(2H)-1,4-benzoxazin-3(4H)-one-hex-hex.

Additionally, the malted wheat or the leachate thereof described herein may further comprise:

(iii) a compound selected from the group consisting of 2,4-dihydroxy-(2H)-1,4-benzoxazin-3(4H)-one or a hexose derivative thereof, benzoxazoline-2-one, and any combination of the foregoing compounds, wherein the concentration of (iii) is higher as compared to the corresponding non-malted wheat.

The consumable product described herein may comprise a concentration of any one of the compounds of (ii) that is higher than the concentration of any one of the compounds of (iii). Alternatively, the concentration of a combination of the compounds of (ii) may be higher than the concentration of a combination of the compounds of (iii).

It will be appreciated that the benzoxazinoid compound 2,4-dihydroxy-(2H)-1,4-benzoxazin-3(4H)-one is a benzoxanoid hydroxamic acid which may be called DIBOA. Further, as used herein benzoxazoline-2-one is a benzoxazinoid which is a benzoxazolinone that may be denominated BOA.

The consumable product described herein comprises malted wheat and/or a leachate thereof in an amount sufficient to induce endogenous production of antisecretory factor (AF) protein and/or fragments thereof in a subject after consumption. The specific amount of the consumable product may be adjusted depending on the condition to be treated. The skilled person may determine the amount using methods known in the art such as determination of the Antisecretory Factor Complex in an Immunoassay (enzyme-linked immunosorbent assay (ELISA)) described herein, either performed by single antibody ELISA (Johansson et al 2009) or by double antibody ELISA (Lönnroth et al. 2015). In one method, as used in example 1-3, the blood plasma sample is purified by agarose affinity chromatography prior to determination in an ELISA by a monoclonal antibody against AF or a polyclonal antibody against C3c. In the double ELISA method, as disclosed in example 4, the blood plasma was tested without purification in ELISA, using a monoclonal antibody against AF as catching antibody and a polyclonal antibody against C3c as detecting antibody.

It has been found that the process for malting the wheat impacts the properties of the consumable product into which it is incorporated. Importantly, the malting should take place at a low temperature such as from about 5° C. to about 20° C. and subsequent drying should take place at a temperature of 80° C. or less. It will be appreciated that in this document the expression “a temperature of 80° C. or less” means a temperature equal to or less than 80° C. Further, the malting may comprise wet steeping in which the wheat is partly or entirely soaked with water such as soaked with water for one hour or more. Additionally, or alternatively, the wet steeping may involve spraying with water. In this way, the malted heat will comprise the compounds (i) and (ii) described herein and beneficially impact induction of antisecretory factor activity.

Thus, there is provided a consumable product as described herein, wherein the malted wheat is obtained from a process comprising the steps of:
a. wet steeping of wheat at a temperature from about 5° C. to about 20° C.,
b. drying of said wheat,
c. growing at a temperature from about 5° C. to about 20° C.,
d. optionally repeating any one of steps a-c, and subsequent
e. drying of said wheat at no more than 80° C.

Steps a. and/or b. described herein may independently take place at a temperature of about 8° C. or from about 13° C. to about 15° C. Further, the wet steeping may be as described herein. The moisture content after any of the process steps may be from about 35 weight-% to about 50 weight-% based on the total weight of the wheat. For instance, the moisture content may be about 42 weight-% or about 47 weight-%. The process described herein may also be characterized by providing malted wheat involving a very small rootless loss, i.e. loss of small roots on the grains being subjected to the process. The rootless loss may be from about 3% to about 5% such as about 4%. Accordingly, there is provided a consumable product as described herein wherein the rootless loss of the malted wheat is from about 3% to about 5% such as about 4%.

The temperature in the drying step e. is measured as air temperature.

The wheat of the consumable product described herein may be malted wheat in which the wheat is selected from the group of wheat varieties consisting of Kosack, Festival, Stava, and any combination thereof. Alternatively, the wheat variety may be Festival and/or Stava. In general, a wheat comprised in the consumable product described herein may be any wheat variety, as long as it displays similar properties before, during and/or after the malting-process described herein, as the varieties Kosack, Festival and Stava, which are tested in the herein incorporated examples 1-5. In particular, a wheat comprised in the consumable product described herein may be any wheat that after malting, according to the malting process described herein, comprises 2,4-dihydroxy-7-methoxy-(2F)-1,4-benzoxazin-3(4H)-one-hex at a concentration of at least 60 ng/mg and 2,4-dihydroxy-7-methoxy-(2H)-1,4-benzoxazin-3(4H)-one-hex-hex at a concentration of at least 2 ng/mg coarse grained malted wheat, as measured e.g. by the method described in example 5.

The aforementioned wheat may be malted and incorporated into the consumable product described herein either as wheat and/or as leachate of said wheat. It has been found that the aforementioned malted wheat and/or leachate thereof provides a consumable product with attractive antisecretory factor activity.

In one embodiment, the herein disclosed consumable product comprises leachate of malted wheat in an amount sufficient to induce endogenous production of antisecretory factor (AF) protein and/or fragments thereof in a subject after consumption, wherein said leachate of malted wheat comprises 2,4-dihydroxy-7-methoxy-(2H)-1,4-benzoxazin-3(4H)-one hex at a concentration of at least 20.000 ng/ml and 2,4-dihydroxy-7-methoxy-(2H)-1,4-benzoxazin-3(4H)-one hex-hex at a concentration of at least 700 ng/ml. In another embodiment, the herein disclosed consumable product consists of leachate of malted wheat, wherein said leachate of malted wheat comprises 2,4-dihydroxy-7-methoxy-(2H)-1,4-benzoxazin-3(4H)-one hex at a concentration of at least 20.000 ng/ml and 2,4-dihydroxy-7-methoxy-(2H)-1,4-benzoxazin-3(4H)-one hex-hex at a concentration of at least 700 ng/ml.

In another embodiment, the herein disclosed consumable product comprises malted wheat in an amount sufficient to induce endogenous production of antisecretory factor (AF) protein and/or fragments thereof in a subject after consumption, wherein said malted wheat comprises 2,4-dihydroxy-7-methoxy-(2H)-1,4-benzoxazin-3(4H)-one hex at a concentration of at least 60 ng/mg and 2,4-dihydroxy-7-methoxy-(2H)-1,4-benzoxazin-3(4H)-one hex-hex at a concentration of at least 2 ng/mg coarse grained malted wheat.

In another embodiment, the herein disclosed consumable product consists of malted wheat, wherein said malted wheat comprises 2,4-dihydroxy-7-methoxy-(2H)-1,4-benzoxazin-3(4H)-one hex at a concentration of at least 60 ng/mg and 2,4-dihydroxy-7-methoxy-(2H)-1,4-benzoxazin-3(4H)-one hex-hex at a concentration of at least 2 ng/mg.

The consumable product described herein may be food, feed, a food supplement, and/or a nutraceutical. The food or feed may be for human and/or animal consumption. Generally, food is intended for human consumption while feed is intended for animal consumption. The consumable product described herein may be a liquid, a solid and/or a combination thereof. For instance, the liquid may be a beverage. In a further example, the consumable product may be an infusion. When the food or feed is a solid it may be dry or semi-dry.

The food described herein may be a medical food. Additionally, or alternatively, the food described herein may be a FSMP, i.e. a food for special medical purposes. It will be appreciated that a FSMP may be food for individuals who suffer from certain diseases, disorders and/or medical conditions, and/or for people whose nutritional requirements cannot be met by normal foods. In a further example, the food described herein may be a nutraceutical. As used herein, a nutraceutical is a food or feed providing an extra health benefit in addition to basic nutritional value in food or feed. The food and/or food supplement for human consumption may be in the form of a liquid, a solid or a combination thereof. In an example, the food for human consumption may be in the form of a liquid, i.e. a liquid food for humans

The feed described herein may be given to animals such as poultry or livestock animals. The feed for animals may be in the form of a liquid, a solid or a combination thereof. In an example, the feed for animals may be in the form of a liquid, i.e. a liquid feed for animals. Examples of poultry include chickens, hens, ducks, geese, pigeons, quails, turkeys, pheasants and ostriches. Examples of livestock animals include cattle such as cows, horses, donkeys, goats, pigs and sheep. In a further example, animals that can be treated with the consumable product described herein include camels, deer, elks, yaks, lamas, alpacas and water buffalos. In still a further example of animals that can be treated with the consumable product described herein include pets such as dogs, cats, rabbits, guinea pigs and hamsters. In a particular example, the feed described herein is horse feed. In a further example, the feed described herein is pig feed. In still a further example, the feed described herein is dog or and/or cat feed. In still a further example, the feed described herein is fish feed.

Moreover, it will be appreciated that the consumable product described herein may be feed for ruminants such as cows, sheep and/or camels. The feed for ruminants may be in the form of a liquid, a solid or a combination thereof. In an example, the feed for ruminants may be in the form of a liquid, i.e. a liquid feed for ruminants.

In the present context, the term “feed” is used to describe materials of nutritional value fed to animals. Each species has a normal diet composed of feeds or feedstuffs which are appropriate to its kind of alimentary tract and which are economically sensible as well as being nutritious and palatable. Animals such as agricultural animals at pasture often have a diet which is very variable and subject to naturally occurring nutritional deficiencies. The feed disclosed herein may help to remedy or at least alleviate such deficiencies as well as disease, condition and/or symptom brought on by a stressful situation and or environment. The presently disclosed feed can further comprise forage feed, such as hay, ensilage, green chop. i.e. any feed with a high cellulose content relative to other nutrients.

The presently disclosed feed can further comprise feed grain such as cereal and other grains and pulses used as animal feed. The aforementioned feed grain may include wheat, barley, oats, rye, maize, peas, raps, rape seed, rape seed meal, soybean meal, and sorghum.

In a further example, the feed described herein may be provided in pelleted form.

The presently disclosed feed can further comprise feed supplements, i.e. nutritive materials which are feedstuffs in their own right and which are added to a basic diet such as pasture to supplement its deficiencies, such as minerals and aromatics. Feed supplements typically include trace elements and macrofeeds, such as protein supplements.

The consumable product can be a feed supplement in itself.

Albeit the present disclosure mainly is directed to a consumable product in the form of food or feed, it is also envisaged that the consumable product may be administrated to a subject in other ways than oral intake. For instance, the consumable product may be provided in a form making it suitable for topical, ocular, subcutaneous and/or systemic administration.

The food described herein may form part of a functional food. For instance, the functional food may be muesli, bread, biscuits, gruel, oatmeal, grains, flakes, pasta, omelet and/or pancake. In an example, the functional food is a beverage, or a food intended to drink. Alternatively, the functional food is not a beverage, or a food intended to drink but a solid or semi-solid foodstuff

Due to the presence of the malted wheat and/or leachate of malted wheat as described herein, the consumable product such as the food and/or feed possesses properties associated with induction of antisecretory factor (AF) protein and/or fragments thereof such as anti-diarrheal properties and/or anti-inflammatory properties. Consequently, the consumable product may be used in treatment, prevention and/or prophylaxis of abnormal physiological conditions caused by pathologically high levels of body fluid discharge. Additionally, or alternatively, the consumable product may be used in the treatment, prevention and/or prophylaxis of a condition which is responsive to increase of antisecretory factor protein and/or antisecretory protein fragments in the blood of a patient. The condition(s) described herein may be selected from the group consisting of diarrhoea, inflammatory diseases, oedemas, autoimmune diseases, cancer, tumours, leukaemia, diabetes, diabetes mellitus, glioblastoma, traumatic brain injury, intraocular hypertension, glaucoma, lipid raft dysfunction, compartment syndrome, Alzheimer's disease, Parkinson's disease, encephalitis, and Meniere's disease.

The consumable product described herein may be provided in the form of a medicament. Thus, there is provided a consumable product as described herein such as a functional food product and/or a pharmaceutical product for use as a medicament.

The present disclosure further provides a method of treatment, prevention and/or prophylaxis of an abnormal physiological condition caused by pathologically high levels of body fluid discharge in a patient in need thereof by feeding said patient a sufficient amount of a consumable product according to the present invention.

Herein is thus provided a method of treatment, prevention and/or prophylaxis of a condition responsive to increase of levels of antisecretory factor protein and/or antisecretory protein fragments in the blood of a patient by feeding said patient a sufficient amount of a consumable product according to the present invention.

In one embodiment, the disclosure provides a method of treatment, prevention and/or prophylaxis of a condition, wherein said condition is selected from the group consisting of diarrhoea, inflammatory disease, oedema, autoimmune disease, cancer, tumor, leukaemia, diabetes, diabetes mellitus, glioblastoma, traumatic brain injury, intraocular hypertension, glaucoma, lipid raft dysfunction, compartment syndrome, Alzheimer's disease, Parkinson's disease, encephalitis, and Meniere's disease.

Further, a consumable product according to the prevention invention can be for use in the manufacturing of a pharmaceutical composition for use in the treatment, prevention and/or prophylaxis of an abnormal physiological condition caused by pathologically high levels of body fluid discharge, and/or for use in the treatment of a condition responsive to increase of levels of antisecretory factor protein and/or antisecretory protein fragments in the blood of a patient.

Thus, the present disclosure also discloses the use of a consumable product according to the present invention for use in the manufacturing of a pharmaceutical composition for use in treating and/or preventing of a condition, wherein said condition is selected from the group consisting of diarrhoea, inflammatory disease, oedema, autoimmune disease, cancer, tumour, leukaemia, diabetes, diabetes mellitus, glioblastoma, traumatic brain injury, intraocular hypertension, glaucoma, lipid raft dysfunction, compartment syndrome, Alzheimer's disease, Parkinson's disease, encephalitis, and Meniere's disease.

The present disclosure will be further explained hereinafter by means of non-limiting examples and with reference to the appended drawings.

### EXAMPLES

The wheat used in the examples herein was purchased from Lantmannen, Sweden. The Kosack wheat was Kosack WW 27084. The Stava wheat was Stava WW 40253. The Festival wheat was Festival wheat SW 95594.

### Example 1

Wheat leachates from Hallfreda wheat, Stava wheat, Festival wheat, Brons wheat and Ceylon wheat were prepared and given to rats as described below.

### Preparation of Wheat Leachate

The wheat grains of malt wheat and control wheat were coarse-grained in a laboratory mill. 40 ml of boiling water was added to 10 g of ground wheat in a 100 ml bottle. The bottle was shaken and allowed to stand for one hour. Thereafter, 10 ml of boiling water was added, and the bottle was placed for one hour in a water bath containing boiling water. Thereafter, the bottle was allowed to cool, and the bottle content was filtrated through a small-meshed nylon filter resulting in 30 ml leachate. The filtrate was centrifuged at 15000 rpm for 20 minutes in a centrifuge operating at 10° C. temperature to provide leach water. The leach water was diluted six times with water, and this diluted leach water was given to the rats. Male Sprague-Dawley rats of body weight 250±20 g (Harlan Laboratories, Boxmeer, Netherlands) housed in a controlled environment were used. The control rats were given tap water the test rats were given leachate of wheat or wheat malt. In addition, all rats received ordinary rat pellets.

In an upscaled preparation protocol, 400 ml of boiling water was added to 100 g of ground wheat in a 1000 ml bottle. The bottle was shaken and allowed to stand for one hour. Thereafter, 100 ml of boiling water was added, and the bottle was placed for one hour in a water bath containing boiling water. Thereafter, the bottle was allowed to cool, and the bottle content was filtrated through a small-meshed nylon filter resulting in 300 ml leachate.

If the leach water was to be analyzed by HPLC/mass spectrometry it was subjected to removal of starch and particles. The supernatant was then frozen overnight, thereafter thawed and again centrifuged at 15000 rpm for 20 minutes, frozen, thawed and centrifuged at 15000 rpm for 20 minutes followed by sterile filtration.

### Feeding of Rats

The wheat malt leachate described above was frozen in 50 ml portions and diluted 1:6 with water each day during the experiment. The control group received tap water. The drinks or the water control were given in excess over 1-14 days before testing of antisecretory activity. After 14 days, blood samples were drawn by heart puncture in tubes with heparin to prevent coagulation.

As previously described, AF1 was affinity purified from blood plasma on small agarose columns and concentration determined by immunoassay (Johansson et al., 2009). In short, 6 ml of the 1:1 diluted plasma samples (described above) were run through 3 ml Sepharose 6B columns, washed twice with phosphate buffered saline (PBS, 0.05 M phosphate, 0.15 M NaCl, pH 7.2) and subsequently eluted with 1 M α-methyl-glycoside. The purified samples were titrated in a 96-well plate, coated over-night, detected by the 3H8 monoclonal mouse IgM antibody against AF1 or PBS as control, and finally developed with a secondary antibody bound to alkaline phosphatase (AP). After absorbance reading at 405 nm, the result was given as reversed titer. The 3H8 antibody recognize the AF sequence responsible for the antisecretory activity (Johansson et al. 2009).

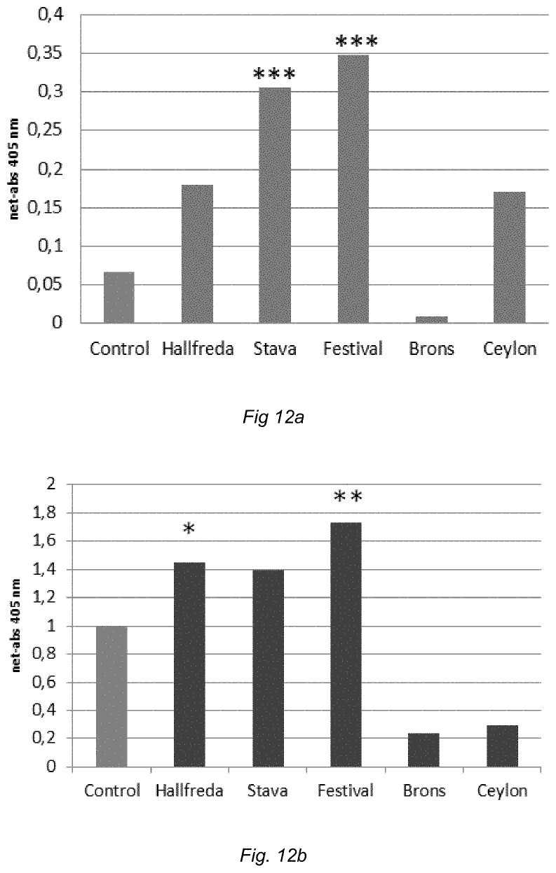

Tables 1 and 2 below show the amounts of AF in blood plasma of rats drinking leachate of malt of different origin corresponding to FIG. 12; The content of AF in blood plasma from the various malt leachates was measured with ELISA using a single monoclonal antibody against AF (FIG. 12a) and a polyclonal antibody against complement C3 (FIG. 12b) (Johansson et al., 2009). The AF and C3 values are given as absorbance at 405 nm. The AF value was significantly higher in leachate of Stava and Festival wheat malt compared to control rats given tap water (p<0.01, resp, 5 animals per group). The C3 value was significantly higher in Festival and Hallfreda wheat malt.

TABLE 1
amounts of AF in blood plasma

| Example | | | | | | |
|---|---|---|---|---|---|---|
| Number | Control | Hallfreda | Stava | Festival | Brons | Ceylon |
| 1 | 0 | 0.168 | 0.394 | 0.427 | 0 | 0.227 |
| 2 | 0.169 | 0.234 | 0.224 | 0.335 | 0 | 0.221 |
| 3 | 0.105 | 0.298 | 0.327 | 0.396 | 0.019 | 0.219 |
| 4 | 0 | 0.201 | 0.306 | 0.248 | 0 | 0.185 |
| 5 | 0.058 | 0 | 0.277 | 0.332 | 0.026 | 0 |
| Average | 0.0664 | 0.1802 | 0.3056 | 0.3476 | 0.009 | 0.1704 |
| T-test | | 0.092016 | 0.000518 | 0.000233 | | 0.090195 |

TABLE 2
C3 values

| Example | | | | | | |
|---|---|---|---|---|---|---|
| Number | Control | Hallfreda | Stava | Festival | Brons | Ceylon |
| 1 | 1.009 | 1.416 | 1.03 | 1.685 | 0.311 | 0.271 |
| 2 | 1.008 | 1.376 | 1.745 | 1.628 | 0.184 | 0.5 |
| 3 | 1.16 | 1.707 | 1.853 | 2.16 | 0.164 | 0.134 |
| 4 | 1.232 | 1.537 | 1.623 | 1.806 | 0.53 | 0.112 |
| 5 | 0.609 | 1.202 | 0.741 | 1.389 | 0.009 | 0.473 |
| Average | 1.0036 | 1.4476 | 1.3984 | 1.7336 | 0.2396 | 0.298 |
| T-test | | 0.011765 | | 0.002301 | | |

### Example 2

### Preparation of Extraction Mix Solution

The preparation of extraction solution mixture has been described in detail elsewhere (Savolainen et al., 2016). In brief, the stable isotope labelled chemicals were prepared in either Milli-Q H2O or liquid chromatography mass spectrometry (LC-MS) grade methanol as 500 ng/μL stock solutions. Chemicals included 13C5-proline (H2O), 2H4-succinic acid (H2O), 13C5,15N-glutamic acid (H2O), 13C4-α-ketoglutarate (H2O), 13C12-sucrose (H2O), 13C6-glucose, 2H4-Putresine, 13C4-hexadecanoic acid (H2O), 2H6-salicylic acid (methanol), 1,2,3-13C3-myristic acid (methanol), 2H7-cholesterol (methanol). The final concentration of these internal standards in the extraction solution was 0.0625 ng/μL in MeOH:H2O (90:10 v/v). All chemicals were from Cambridge Isotope laboratories (Tewksbury, Mass.).

### Sample Preparation

In total, 6 wheat samples with and without prior malting were analyzed.

Wheat was processed in a micro malting facility (Lahtis. Finland) as described in Table 3.

TABLE 3
Malting of cereals
MALTING PROCESS

| Steeping | | 7w + 17d + 7w = 31/17h | | | | | |
|---|---|---|---|---|---|---|---|
| program | | (w: wet, d: dry) | | | | | |
| Wet/dry | oC/oC | 13/15 | | | | | |
| steeping | | | | | | | |
| temperature | | | | | | | |
| Moisture | % | 32.1 | 31.3 | 33.0 | 31.9 | 31.4 | 31.8 |
| after 1. Wet | | | | | | | |
| steep | | | | | | | |
| Moisture | % | 41.8 | 40.8 | 42.7 | 41.0 | 40.3 | 42.2 |
| after | | | | | | | |
| steeping | | | | | | | |
| Germination | | 5 days/15oC | | | | | |
| program | | | | | | | |
| Spraying | | 1 | 1 | 1 | 1 | 1 | 1 |
| day | | | | | | | |
| Moisture | % | 47 | 47 | 47 | 47 | 47 | 47 |
| after | | | | | | | |
| spraying | | | | | | | |
| Germination | % | 99 | 97 | 99 | 96 | 99 | 95 |
| 1 days | | | | | | | |
| Green malt | % | 43.6 | 43.9 | 44.1 | 44.8 | 43.7 | 44.4 |
| moisture | | | | | | | |
| Kilning | | PM | | | | | |
| program | | | | | | | |
| Respiration | % | 2.8 | 2.8 | 3.0 | 3.0 | 2.3 | 2.7 |
| losses | | | | | | | |
| Rootlet | % | 4.1 | 3.9 | 4.0 | 4.5 | 3.5 | 3.7 |
| losses | | | | | | | |
| Total losses | % | 6.9 | 6.7 | 6.9 | 7.5 | 5.8 | 6.3 |

Growing was performed at a thermostatic temperature with streaming humid air. After along, cool period of germination, at less than 17° C. (62.6° F.), the fully modified malt was well dried to about 8% in a cool, fast airflow before being cured with slowly rising temperatures up to 70-85° C. (158-185° F.). The very pale products, which have no trace of caramel or malanoidin coloration had very weak aromas.

### Example 3

The samples represented the following varieties: Kosack (K), Festival (F), Stava (S), Hallfreda (H), Brons (B) and Ceylon (C). Wheat sample extracts were thawed at room temperature for 30 min and a 100 μL aliquot of each sample was transferred into a 1.5 ml microcentrifuge tube. Cold extraction solution (900 μL) was mixed with samples using a multi-tube vortexer (VWR International, Inc) for 10 min and incubated at 4° C. for 2 h. The mixtures were centrifuged for 12 min at 13000 rpm at 4° C. The supernatant from each sample was kept in refrigerator at 4° C. until they were injected on the LC-MS instrument. Each wheat sample was prepared in triplicates. Quality control samples (QC) were achieved by pooling aliquots of all the study wheat samples (i.e. 6 varieties with and without treatments) and were used to monitor the stability and functionality of the system throughout the instrumental analyses.

### Analytical Protocol of Untargeted LC-MS Metabolomics

Wheat extract samples were analyzed by LC-Q-TOF mass spectrometry-MS (Agilent Technologies 6550 iFunnel Q-TOF LC/MS, United States). Sample solution (5 μL) was injected for reversed-phase (RP) chromatographic analyses using both positive and negative electrospray ionization modes. Separation was performed using an Acquity UPLC High Strength Silica T3 column (2.1×100 mm, 1.8 um; Waters) at 45° C. The mobile phase was delivered at 400 μL/min and consisted of eluent A (water, Milli-Q purified; Millipore) and eluent B (methanol, Sigma-Aldrich), both containing 0.04% (v/v) of formic acid (Sigma-Aldrich), delivered in a profile: 0-10.5 min 100% B, 10.51-15 min: 5% B. The dual electrospray ionization source (ESI) was operated using the following conditions: Drying gas (nitrogen) temperature of 175° C. and flow of 10 L/min, nebulizer pressure of 45 PSI, capillary voltage of 3500 V, fragment or voltage of 175 V, and a skimmer of 65 V. For data acquisition, a 2-GHz extended dynamic range mode was used, and the instrument was set to acquire over the mass range of m/z 50-1700. Data were collected in centroid mode at an acquisition rate of 1.67 spectra/s with an abundance threshold of 200 counts. Continuous mass axis calibration was performed by monitoring two reference ions, m/z 121.050873 and m/z 922.009798 for positive mode and m/z 112.98558700 and 966.000725 for negative mode, from an infusion solution throughout the runs.

All the wheat samples were analyzed randomly in one batch. Two blank samples and one priming quality control sample provided by the Chalmers Mass Spectrometry Infrastructure were injected before the analytical sequence. Two pooled QCs described as above were injected at the beginning and end and as every 10th injection throughout the sequence.

### Data Pre-Processing

Raw data files from RP (ESI+), RP (ESI−) were converted to mzML format using ProteoWizard msconvert (Chambers et al., 2012). Data deconvolution was performed with xcms, a freely available software under open-source license, implemented in R (Smith et al., 2006). Specifically, feature detection in each chromatogram was performed using the centWave algorithm implemented in the xcmsSet function and obiwarp was applied for retention time correction. The term ‘feature’ refers to a mass spectral peak, i.e. a molecular entity with a unique mass-to-charge ratio and retention time as measured by an LC-MS instrument. Parameters were the values suggested by xcms online (https://xcmsonline.scripps.edu) and from recently relevant publications (Stanstrup et al., 2013; Zhu et al., 2013; Ganna et al., 2016; Shi et al., 2018). Parameters were: peakwidth=c(10, 60), ppm=15, prefilter intensity (3, 1000), bandwidth (2), mzdiff (0.01). Quality of data acquisition and processing was examined by visualization of the total ion chromatogram and the base peak chromatogram for each sample, extracted-ion chromatograms for multiple features, and assessment of differences between adjusted and raw retention times per sample. Within-batch signal intensity normalization was performed using R package ‘batchcorr’(Brunius et al., 2016). Features passing a QC test (CV<0.3) were determined as qualified features and were further subjected to statistical analyses. In total, 3511 and 3809 features were retained after a stringent normalization procedure for RP (ESI+) and RP (ESI−), respectively. Missing values were imputed by using random forest algorithm implemented in R package ‘missForest’ (Stekhoven and Bühlmann, 2012).

### Statistical Analysis

To identify features that predicted antisecretory factor activity in wheat samples, data obtained from RP (ESI+) and RP (ESI−) were processed independently. Regression modelling was performed using a random forest algorithm incorporated into a repeated double cross-validation framework with unbiased variable selection (Shi et al., 2018). The antisecretory activity rank for various wheat varieties measured by dividing into four groups corresponding to their AF inducing activity with group 1 as the lowest activity and 5 as the highest activity. Group 1) Brons and Ceylon, group 2) Hallfreda group 4) Stava and Festival, Group 5) Kosack and used as dependent variable in the model. Correlations between selected features were examined by Pearson correlation coefficients. For each feature selected by multivariate modelling, correlation between feature intensity and the antisecretory factor activity rank was assessed using linear regression. Moreover, differences in antisecretory factor activity related metabolites between treated and corresponding untreated samples for each wheat sample were tested using Wilcoxon signed-rank test. Data analyses and result visualization were performed in R software version 3.4 using the packages MUVR, Ime4, corrplot.

### Metabolite Identification

Metabolite identification was accomplished based on accurate mass and MS/MS fragmentation matched against online databases (i.e. Metlin, FooDB and MassBank) or the literature (De Bruijn et al., 2016; Hanhineva et al., 2011; Koistinen et al., 2018). The confidence level of annotation was categorized according to the Metabolomics Standard Initiative (MSI) (Sumner et al., 2007).

### Results

The following compounds were found to have a good prediction for antisecretory factor activity and to correlate positively with antisecretory factor activity: 2,4-dihydroxy-7-methoxy-(2H)-1,4-benzoxazin-3(4H)-one (i.e. DIMBOA) or a hexose sugar or glc derivative thereof. Further, when the wheat as described herein (i.e. Kosack wheat (K), Festival wheat (F), Stava wheat (S), Hallfreda wheat (H), Ceylon wheat (C), Brons wheat (B)) was subjected to the malting process described herein this resulted in an increased concentration of the compounds as compared to the corresponding non-malted wheat.

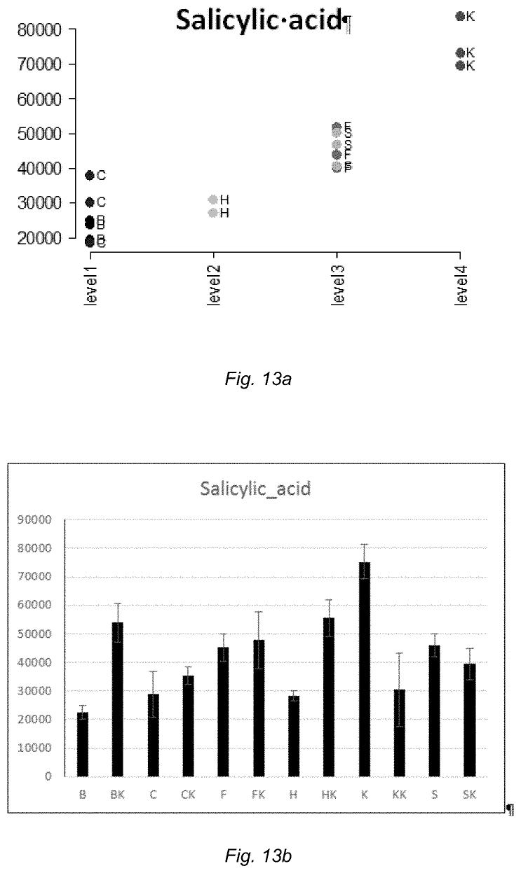

Moreover, salicylic acid was found to have a good prediction for antisecretory factor activity and to correlate positively with antisecretory factor activity. It was found that subjecting the wheats Festival, Stava and Kosack to the malting process described herein did not substantially affect the concentration of salicylic acid or increased the concentration of salicylic acid as compared to the corresponding non-malted wheat. Further, it was found that subjecting the wheats Brons, Ceylon and Hallfreda to the malting process described herein resulted in a decrease in the concentration of salicylic acid as compared to the corresponding non-malted wheat (see FIGS. 13a and b).

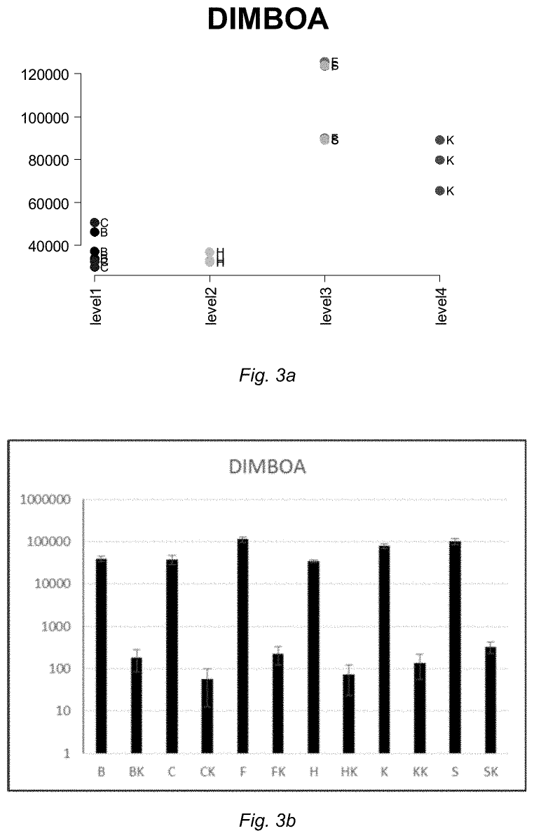

FIG. 3a shows the correlation between the concentration of DIMBOA and antisecretory factor activity. FIG. 3b shows that the concentration of methyl DIMBOA increases when the wheat is subjected to the malting process as described herein as compared to the corresponding non-malted wheat.

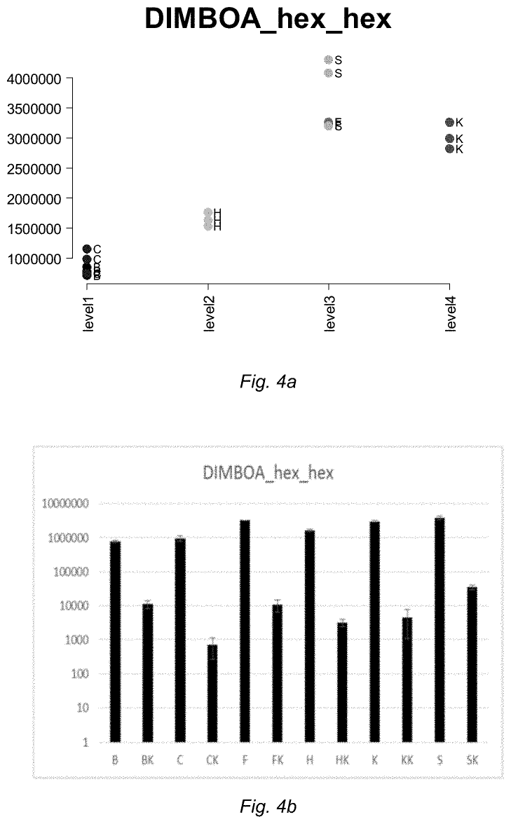

FIG. 4a shows the correlation between the concentration of DIMBOA_hex_hex and antisecretory factor activity. FIG. 4b shows that the concentration of methyl DIMBOA_hex_hex increases when the wheat is subjected to the malting process as described herein as compared to the corresponding non-malted wheat.

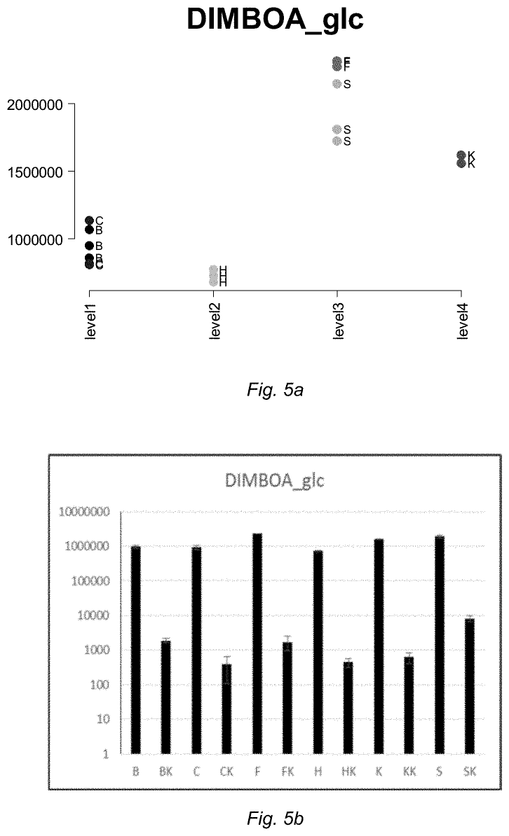

FIG. 5a shows the correlation between the concentration of DIMBOA_glc and antisecretory factor activity. FIG. 5b shows that the concentration of methyl DIMBOA_glc increases when the wheat is subjected to the malting process as described herein as compared to the corresponding non-malted wheat.

FIG. 13a shows the correlation between the concentration of salicylic acid and antisecretory factor activity. FIG. 13b shows that the concentration of salicylic acid increases in Stava and Kosack wheat when the wheat is subjected to the malting process as described herein as compared to the corresponding non-malted wheat.

The following compounds were found to have a good prediction for antisecretory factor activity and correlate negatively with antisecretory factor activity:

benzoxazolinone (i.e. BOA), 2,4-dihydroxy-(2H)-1,4-benzoxazin-3(4H)-one (i.e. DIBOA) or a hexose derivative thereof.

Further, when the wheat as described herein (i.e. Kosack wheat (K), Festival wheat (F), Stava wheat (S), Hallfreda wheat (H), Ceylon wheat (C), Brons wheat (B)) was subjected to the malting process described herein this resulted in an increased concentration of the compounds as compared to the corresponding non-malted wheat.

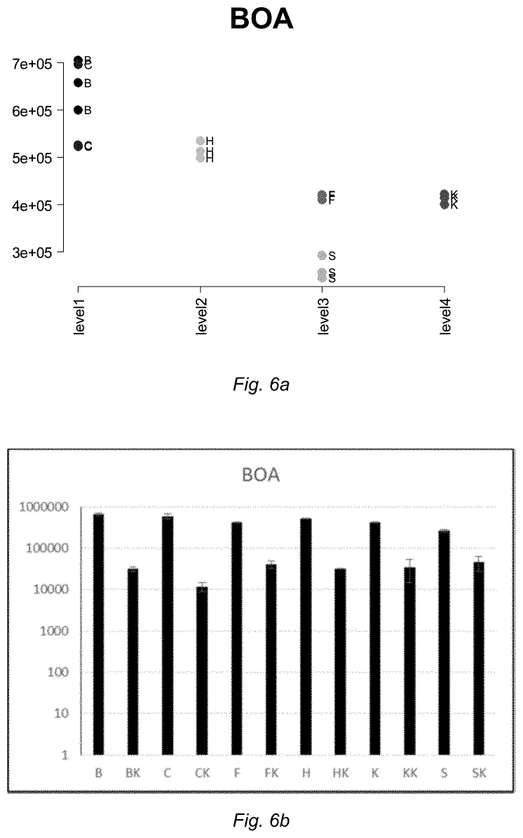

FIG. 6a shows the correlation between the concentration of BOA and antisecretory factor activity. FIG. 6b shows that the concentration of BOA increases when the wheat is subjected to the malting process as described herein as compared to the corresponding non-malted wheat.

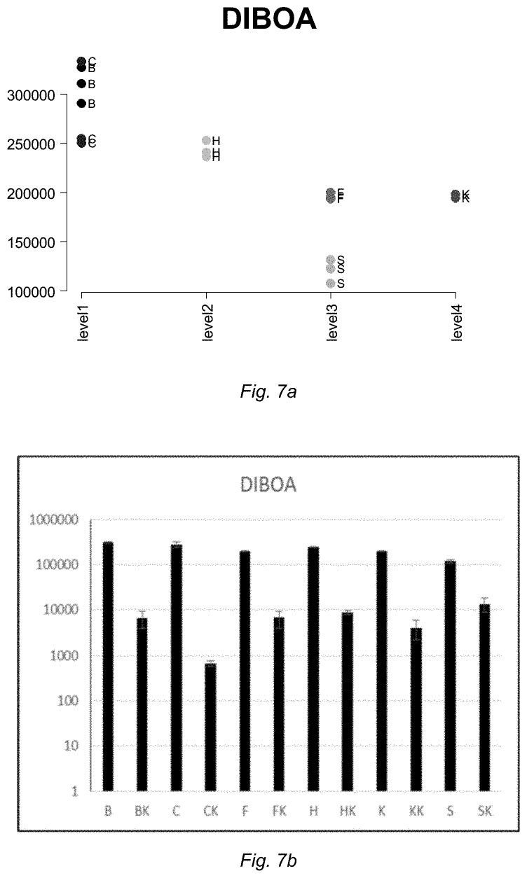

FIG. 7a shows the correlation between the concentration of DIBOA and antisecretory factor activity. FIG. 7b shows that the concentration of DIBOA increases when the wheat is subjected to the malting process as described herein as compared to the corresponding non-malted wheat.

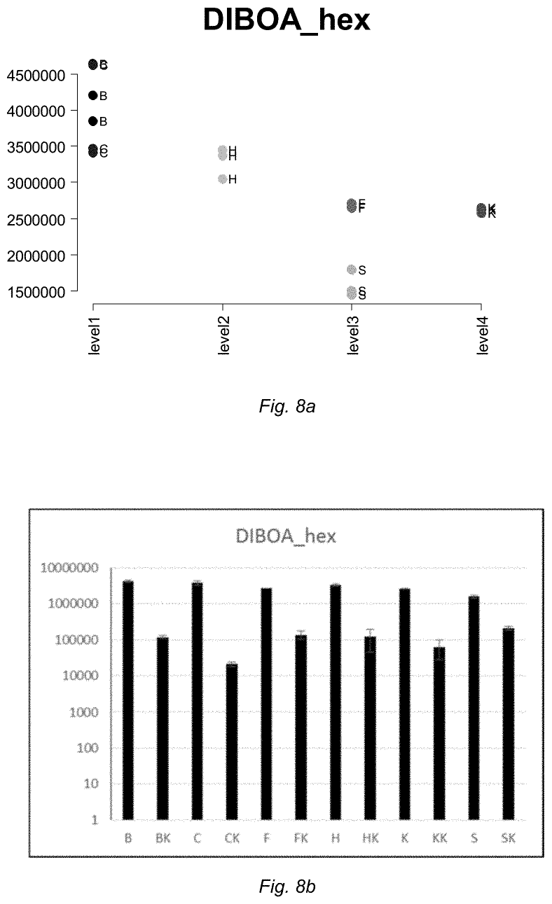

FIG. 8a shows the correlation between the concentration of DIBOA_hex and antisecretory factor activity. FIG. 8b shows that the concentration of DIBOA_hex increases when the wheat is subjected to the malting process as described herein as compared to the corresponding non-malted wheat.

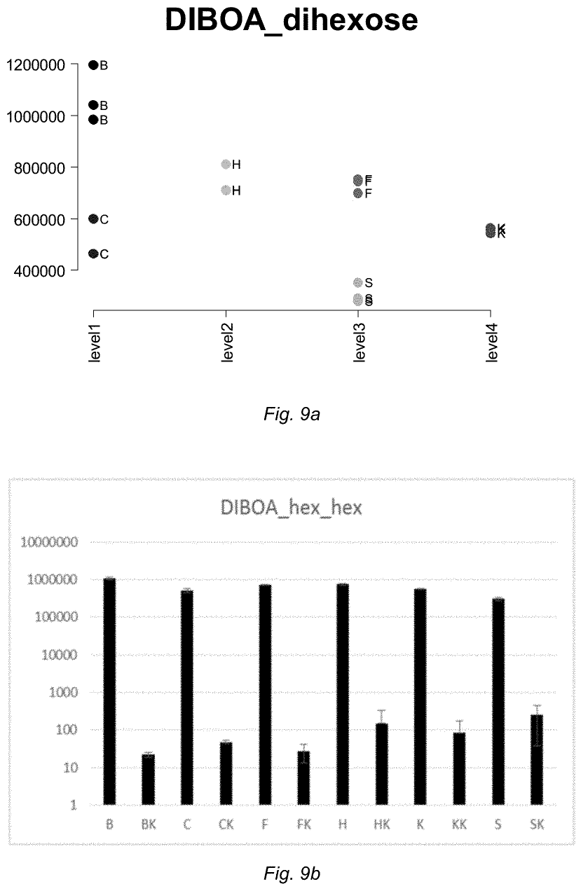

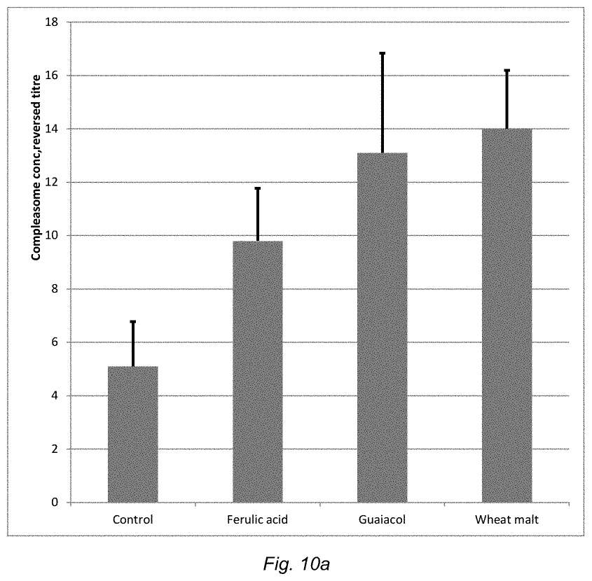

FIG. 9a shows the correlation between the concentration of DIBOA_dihexose and antisecretory factor activity. FIG. 9b shows that the concentration of DIBOA_hex_hex increases when the wheat is subjected to the malting process as described herein as compared to the corresponding non-malted wheat.

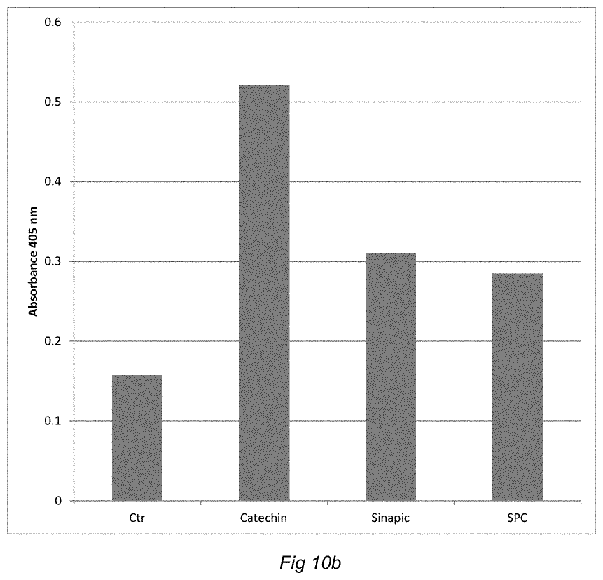

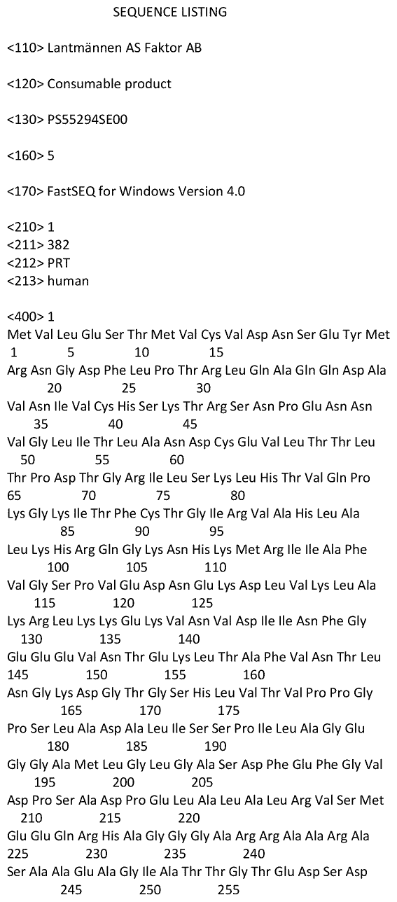

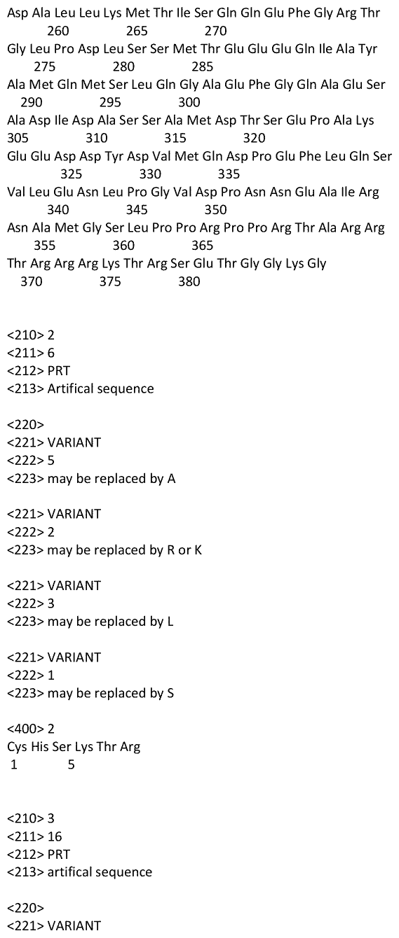

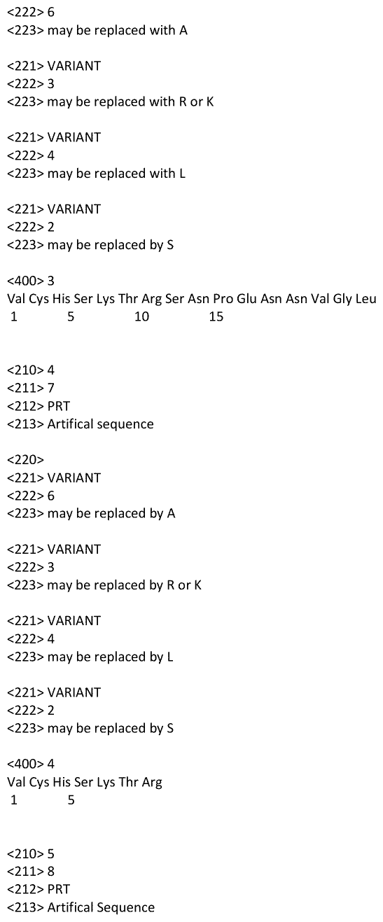

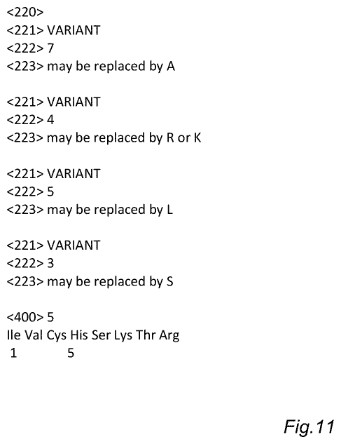

It will be appreciated that in FIGS. 6a-15a the y-axis indicates the detector response of the metabolites being examined, and the x-axis indicates the antisecretory factor activity such that level 1 is lower than level 4.

Further, it will be appreciated that DIBOA_dihexose and DIBOA_hex_hex may be used interchangeably.

In this document, the letter “K” used in combination with the letters for the wheats (Kosack (K), Festival (F), Stava (S), Hallfreda (H), Brons (B) and Ceylon (C)) is understood to mean “control”, i.e. that the wheat was not malted. Thus, KK means non-malted Kosack wheat FK means non-malted Festival wheat, SK means non-malted Stava wheat, HK means non-malted Hallfreda wheat, BK means non-malted Brons wheat, CK means non-malted Ceylon wheat.

### Example 4

### Material and Methods

### Chemicals

Clostridium difficile toxin A (CDA toxin) was produced as previously described (Torres et al. 1991). Cholera toxin was obtained from List Biological Laboratories.

Monoclonal IgM antibodies against AF/RPN10 were produced as previously described. Polyclonal antibodies against complement factor C3 were obtained from Dako DK (www.dako.com item A0062). Secondary antibodies, alkaline phosphatase conjugated goat anti-rabbit IgG, and goat anti-mouse IgM were obtained from Jackson ImmunoResearch. Folin & Ciocalteu's phenol reagent and the pure phenols were obtained from Sigma-Aldrich, and the solvents for HPLC chromatography were obtained from Merck.

### Cereal

Kosack wheat was processed in a micro malting facility (Danbrew Ltd) as described in Table 4.

TABLE 4
Malting of cereals

| | Wheat | |
|---|---|---|
| Process | Hours | Temp ° C. |
| Steeping 1 | 6 | 14.5 |
| Growing 1 | 15 | 13.5 |
| Steeping 2 | 5 | 14.5 |
| Growing 2 | 120 | 8.5 |
| Drying | 10 | 50 |
| | 8 | 65 |
| | 8 | 80 |

Growing was performed at a thermostatic temperature with streaming humid air.

The contents of sugars and free amino acids were analysed before and after malting by Eurofins Sweden, Lidköping. Muesli and rusk were baked containing 30% and 36%, respectively of the malted grain.

### Setup of the Animal Experiment

The study design was approved by the Ethics Committee of the University of Gothenburg (122-2002, EC Directive 86/609/EEC). The animal experiment used male Sprague-Dawley rats of body weight 250±20 g (Harlan Laboratories, Boxmeer, Netherlands) housed in a controlled environment.

In one set of experiments, the rats were given different cereal feed in the form of pellets. The grain, rusk, or muesli of Kosack wheat was suspended in water together with ordinary rat pellets in a 1:5 proportion calculated from the dry weight. The feed was baked in cylinders and dried in an oven before chopping into 10×20 mm pellets. The rats were fed for 7 days before testing of AF activity.

In another set of experiments, the rats were given water extracts of the cereals or purified phenols in their drinking water. The malted Kosack wheat was milled to flour, of which 200 g was put in a glass flask and 800 ml boiling water was successively mixed with the flour. After 3 hours of slow cooling to room temperature, the soak-water was filtered through a nylon filter and centrifuged for 30 min at 12000×g. The supernatant was frozen in 50 ml portions and diluted 1:6 with water each day during the experiment. Freeze-dried purified fractions of wheat extracts were dissolved in the drinking water. Pure phenols were dissolved in the drinking water in 5 μM concentration. The drinks or the water control were given in excess over 1-14 days before testing of antisecretory activity.

### Intestinal Loop Test of Antisecretory Activity

The antisecretory activity was determined by the ligated loop assay in rats (n=4-6) using cholera toxin or CDA toxin as a secretagogue (Lange, 1982) One loop of about 10 cm in length was ligated in the jejunum under isoflurane anaesthesia. The loop was challenged with 1.0 ml of cholera toxin or CDA toxin in physiological phosphate buffer (0.15 M NaCl, 0.05 M Na₂HPO₄, pH 7.5), the toxin concentration being titrated to give 90% of the maximal secretion (normally 1-3 μg toxin). Net fluid secretion (mg/cm) was estimated by subtracting the weight of a control loop from that of the experimental animal loop. In order to quantify the increase of activity during purification, antisecretory Units were introduced, with one Unit being the amount that caused 50% inhibition of fluid secretion (Bjorck et al., 2000).

### Compleasome Assay in Blood

Rats were given leachate from malted wheat or pure phenols in the drinking water as described above. After 14 days, blood samples were drawn as described elsewhere (Bjorck et al., 2000 and Lönnroth et al., 2016) and the antisecretory activity in blood was estimated by performing a sandwich enzyme-linked immunosorbent assay (ELISA) for detection of compleasomes (proteasome/complement complexes) in blood plasma as described elsewhere (Lonnroth et al., 2016). A monoclonal antibody (mab) against antisecretory factor was used as catching antibody and a polyclonal antibody against complement factor 3c as detecting antibody. The titre was determined after development by a secondary antibody coupled to alkaline phosphatas.

Purification of Phenols from Wheat

The leachate prepared from the malted wheat was further purified and tested for antisecretory activity in rats. The activity was related to the dry weight of each fraction. The clear leachate was ultra-filtrated through a Dia-Flo PM10 filter (Millipore Corporation, cut off 10000) and run through a column with hydrophobic resin (Amberlite XAD-2) to which the cereal phenols were attached (Martos et al., 2000). The column was eluted with water of increasing temperature up to 100° C. The elute between 60-100° C. was cooled to room temperature and run through a sterile filter. The filtrate was run through reversed phase HPLC using a preparative C18 column (SP 250/21 Nucleosil 100-7). The absorbed material was eluted with a linear 0-100% water-acetonitrile. The fractions eluting at 20-27% acetonitrile contained the highest activity and was further purified in a smaller high resolution HPLC (C18, 5 μm) column which was eluted with 50% methanol/water plus 0.5% acetic acid in a linear flow of 150 μL/min which was coupled to a electrospray mass spectrometer.

### Mass Spectrometry

The purified HPLC fraction was analysed in a positive electrospray (ZabSpec FPD, VG Analytical Micromass) in order to obtain the molecular mass of the active phenol.

The quantitative assay of ferulic and vanillic acid in the wheat leachate was performed at the Swedish Metabolomics Center, Swedish University of Agricultural Sciences, Umeå. The sterile filtrated leachate was applied on a HPLC HSS T3 (2.1×100 mm, Waters) with C13-labelled ferulic, sinapic acid, catechin and vanillic acid as internal standards, eluted with a linear gradient of 0-100% water/acetonitrile with 0.1% formic acid, and analysed in an Agilent 6490 triple quadrupole mass spectrometer (Hugin).

### Total Phenols

The concentration of total phenols in malt and control wheat leachate was estimated with Folin & Ciocalteu's phenol reagent using ferulic acid as standard. In short, a 20-μL aliquot of the extracts or a ferulic acid standard solution was pipetted into a cell of a 96-cell microplate, followed by the addition of 100 μL of 0.4 N Folin & Ciocalteu's phenol reagent and 80 μL of 0.94 M Na₂CO₃. The plate was covered with a plastic plate cover and allowed to develop colour for 5 min at 50° C. The absorbance was read at 765 nm using a microplate spectrophotometer (Soft Max Pro).

### Statistics

Graphs were constructed using Excel 2010. A one-way Student's t-test was used for comparing mean values in order to calculate the p-values of significance. Thus, statistics are presented using the mean value±standard error of the mean unless otherwise stated.

### Results

### Antisecretory Activity in Cereal Feed

The pellets containing malted or control wheat or ordinary rat feed were given to rats for 7 days before antisecretory activity was tested in an intestinal loop test. The processed wheat showed antisecretory activity whereas the ordinary wheat and control feed produced no detectible antisecretory activity (Table 5). Various wheat flour products were tested for antisecretory activity with the same method, and both rusks and muesli baked from the processed wheat showed good activity.

TABLE 5
Cereal products were mixed with standard rat feed in a
1:5 ratio. After 7 days the antisecretory activity was tested in a
ligated intestinal loop test. One AF unit was defined as the
capacity to cause 50% inhibition of cholera toxin-induced secretion.

| | Anti cholera | Anti CDA toxin* |
|---|---|---|
| Product | secretion, % | secretion, % |
| Wheat grain, control | 0 | 0 |
| Wheat grain, 100% malt | 82 | 74 |
| Wheat rusk, 30% malt | 49 | — |
| Wheat muesli, 36% malt | 63 | — |

*Toxin A from Clostridium difficile

### Purification of Antisecretory Factor-Inducing Components

Malted Kosack wheat or control wheat was ground and soaked in boiling water, and after separation by centrifugation the supernatant was used for further purification of the active components. Ultrafiltration (cut off 10 kDa) of the leachate revealed that the activity did not reside in the macromolecular retained fraction. The filtrates of the processed and control wheat contained 2.0 mM and 0.4 mM phenols, and the contents of free amino sugars and sugars increased substantially after processing.

The active components were shown to attach to a hydrophobic Amberlite XAD-2 resin which mainly absorbed the phenolic substances in the wheat. The activity eluted with hot water at temperatures between 60-100° C. As seen in Table 6, the activity of the processed wheat was purified from 0.0044 units/g dry weight to 4.5 units/g in the XAD eluate.

TABLE 6
Antisecretory activity, units/g dry weight

| | |
|---|---|
| Soak water | 0.017 |
| XAD eluate 60-100° C. | 4.5 |
| HPLC fraction 1 (15-20% acetonitrile) | 67 |
| HPLC fraction 2 (20-23% acetonitrile) | 333 |

Further purification was achieved on a large preparative C18 HPLC column (Table 2 and supplement). The absorbed material on the C18 resin was eluted with a linear 0-100% water-acetonitrile, resulting in two fractions containing the main activity. The first fraction contained 67 and the second 333 AF units per gram dry-weight.

The fractions eluting at 20-23% acetonitrile contained the highest activity (333 units per g dry weight). Further purification of the latter fraction on an analytical C18 column and analysis with mass spectrometry revealed a peak consisting of guaiacol (supplement). The concentrations in the original leachate of catechin, ferulic acid, sinapic acid and vanillic acid (two of the major wheat phenols with similar structure as guaiacol) were determined separately using isotope-labelled phenols as reference for mass spectrometry. These concentrations were found to be 927±67 and 240±25 μg/L, respectively

### Concentration of Phenols in Leachate of Wheat and Wheat Malt

The concentration of four phenols catechin, ferulic acid, sinapic acid and vanillic acid was quantitated with help of their isotope labelled tracer substances.

As shown in table 7, catechin, ferulic acid and sinapic acid was more than doubled in the malt compared to the control wheat, whereas vanillic acid somewhat decreased.

TABLE 7
Concentration of phenols in leachate of wheat and wheat malt

| | | Ferulic | Sinapic | Vanillic | Total |
|---|---|---|---|---|---|
| | Catechin | acid | acid | acid | phenols |
| | pg/μl | pg/μl | pg/μl | pg/μl | mM |
| wheat | 8 | 562 | 40 | 442 | 0.4 |
| wheat | 306 | 1792 | 195 | 515 | 2.0 |
| malt | | | | | |

### Identification of Active Phenols

Plant phenols with the most similar structures to 2-methoxyphenol (guaiacol) were given to the rats in pure form in drinking water, and the antisecretory activity tested. As seen in Table 8, guaiacol induced antisecretory activity as early as day 1, and the activity increased successively on days 3 and 5. Salicin and resorcinol had a much smaller effect, whereas the fully methylated guaiacol-derivate veratrole had an opposite effect in that it induced secretion. The major phenols in wheat ferulic acid, vanillic acid and quercetin, caused a significant antisecretory activity. Eudesmine containing two fully methylated catechol rings increased secretion in the same way as veratrole. The vanilloid receptor (TRPV1) antagonist capsazepine was as active as guaiacol, having a pronounced AF activity on day 1.

TABLE 8
Antisecretory activity of plant phenols and model phenols.
The substances were given to rats in drinking water at 0.01 mg/ml for 1-5 days, whereupon
fluid secretion was induced in ligated intestinal loops by cholera toxin.

| Substance | Days* | Inhibition, % | AF units** | Formula |
|---|---|---|---|---|
| Guaiacol | 1 3 5 | 46 51 76 | 0.9 ± 0.3 1.0 ± 0.2 1.5 ± 0.2 | 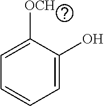 |
| Salicin | 5 | 17 | 0.3 ± 0.1 | 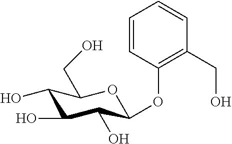 |
| Resorcinol | 5 | 30 | 0.6 ± 0.1 | 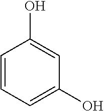 |
| Veratrole | 5 | 21 | secretion | 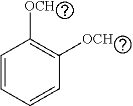 |
| Ferulic acid | 5 | 67 | 1.2 ± 0.2 | 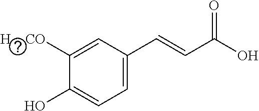 |
| Vanillic acid | 5 | 57 | 1.1 ± 0.1 | 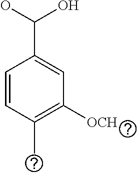 |
| Quercetin | 5 | 42 | 0.8 ± 0.1 | 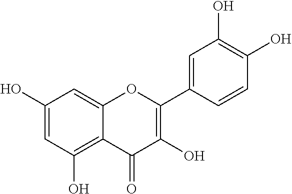 [SMILES: O=c1c(O)c(-c2ccc(O)c(O)c2)oc2cc(O)cc(O)c12] |
| Eudesmine | 5 | 16 | secretion | 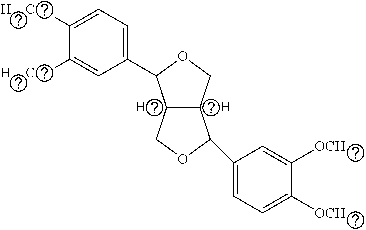 |
| Capsazepine | 1 3 | 38 66 | 0.8 ± 0.1 1.3 ± 0.1 | 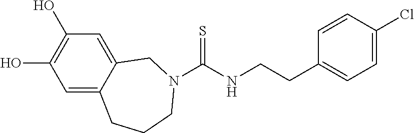 [SMILES: Oc1cc2c(cc1O)CN(C(=S)NCCc1ccc(Cl)cc1)CCC2] |

*Days with substance in the drinking water.
**One AF unit is defined as being equivalent to 50% inhibition of the cholera toxin-induced secretion.

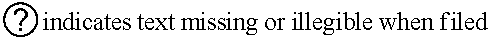

### Induction of Antisecretory Factor in Blood

The capacity of guaiacol, ferulic acid, sinapic acid, catechin and vanillic acid to induce AF in blood was tested using an ELISA. The phenols were given to rats in 5 μM concentration in drinking water, while controls received water during the same period. The AF activity in blood was significantly increased in the animals receiving the four phenols were increased to more than the double concentration. The leachate from wheat malt also showed activity in contrast to the wheat control.

### Conclusion

Extensive malting of wheat induces protection against intestinal secretion and diarrhoea. Three substances in the malt were identified as causing >50% inhibition, namely guaiacol, ferulic acid, sinapic acid and vanillic acid. These substances, all of which have a 2-methyl-catechol structure, were able to induce the AF compleasome in the blood which protects against intestinal secretion. The induction of AF seems to involve the vanilloid receptor TRPV1 in the gut.

### Example 5

### Aim

To develop a targeted quantitative method for DIMBOA, DIMBOA_hex, and DIMBOA_hex_hex molecule features that were selected based on their importance for the effects observed in previous experiments.

### Samples and Standards

100 μL of each sample (i.e. leachates of malted wheat, prepared as described in example 1 above) was extracted with 900 μL of 80% methanol, shaken vigorously for 5 min and incubated at +4° C. for two hours. Thereafter, samples were centrifuged at 4° C. for 10 min at 12 000 g and the supernatant was recovered. DIMBOA and DIMBOA_hex standards were prepared in and diluted with 80% methanol and calibration curves ranged from 10 ng/ml to 10 000 ng/ml. As there was no authentic standard available for DIMBOA_hex_hex it was calibrated by using DIMBOA_hex calibration curve. Samples and standards were analysed in triplicates and the mean was used for calculating analyte concentrations.

### UHPLC-MS/MS Analysis

Samples were measured by using a UHPLC-MS/MS system (ExionLC with AB Sciex QTRAP 6500+) as follows. A Waters HSS T3 column (100×2.1 mm, 1.8 um), held at 45° C., was used to separate the analytes. Mobile phase A was water with 0.04% formic acid and B methanol with 0.04% formic acid. Gradient was as follows: 5% B->100% B in 6 min where held for 2 min before returning to initial conditions. Injection volume was 10 L, total flow rate 0.4 ml/min and the autosampler was kept at 8° C. Mass spectrometry parameters were set as follows: electrospray ionization (−4500 V), GS1 40, GS2 55, CUR 40, CID low and TEM 600. Parameters for monitoring of the analytes are presented in Table 9.

TABLE 9
Multiple reaction monitorinq parameters

| | Q1 | Q3 | | RT |
|---|---|---|---|---|
| Analyte | (Da) | (Da) | CE | (min) |
| DIMBOA | 209.9 | 149.0 | −12 | 3.55 |
| DIMBOA_hex | 372.0 | 149.0 | −30 | 3.40 |
| DIMBOA_hex_hex | 534.0 | 192.0 | −18 | 3.10 |

### Results

We previously reported a metabolite feature (m/z=201.04, retention time (RT)=186 second) putatively annotated as DIMBOA in untargeted metabolomics analysis. However, by matching it against the standard spectrum of DIMBOA, we found that this ion is not from DIMBOA, rather a fragmentation product of DIMBOA_hex. No DIMBOA could be detected using the current targeted analysis method. Our findings put a big question-mark to identification and reporting of DIMBOA in the literature.

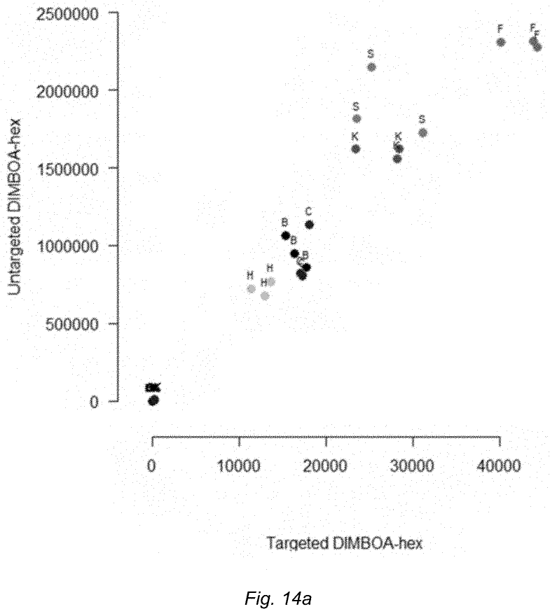

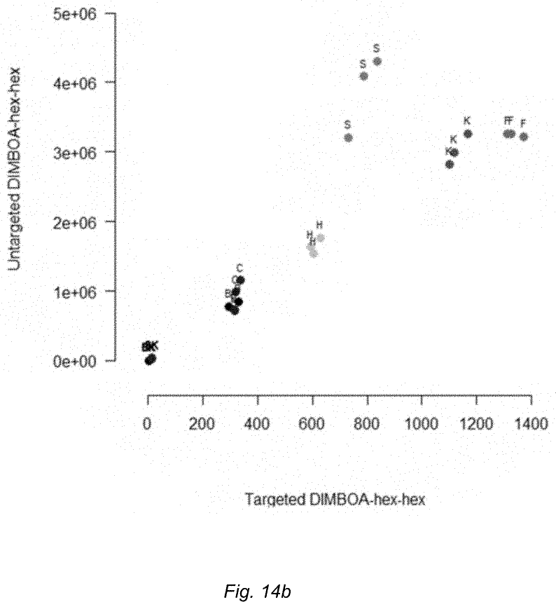

The concentrations of DIMBOA_hex and DIMBOA_hex_hex were determined (Table 10) and compared with semi-quantitative untargeted analysis conducted previously (Table 10 and FIG. 14).

TABLE 10
Concentrations of DIMBOA_hex and DIMBOA_
hex_hex measured by targeted analysis and
untargeted metabolomics conducted previously.

| | Targeted analysis (ng/ml in | | Untargeted analysis (relative | |
|---|---|---|---|---|
| | sample, 10-fold dilution) | | metabolite feature intensity) | |
| Sample | DIMBOA_ | DIMBOA_ | DIMBOA_ | DIMBOA_ |
| Name | hex | hex_hex | hex_hex | hex |
| B | 16413 | 295 | 777546 | 948586 |
| B | 17666 | 315 | 711288 | 857097 |
| B | 15357 | 330 | 851623 | 1065801 |
| BK | 50 | 2 | 15409 | 1973 |
| BK | 57 | 2 | 9447 | 1521 |
| BK | 51 | 2 | 8899 | 2230 |
| BR | NA | NA | 120 | 91 |
| BR | NA | NA | 289 | 23 |
| BR | NA | NA | 13 | 660 |
| C | 16990 | 316 | 736439 | 817444 |
| C | 17237 | 338 | 1149749 | 805724 |
| C | 18093 | 319 | 981834 | 1133914 |
| CK | 5 | 1 | 400 | 16 |
| CK | 5 | 1 | 392 | 681 |
| CK | 4 | 1 | 1282 | 465 |
| F | 40115 | 1313 | 3258530 | 2310341 |
| F | 44291 | 1373 | 3219365 | 2275278 |
| F | 43860 | 1326 | 3261574 | 2315228 |
| FK | 52 | 3 | 11012 | 1738 |
| FK | 45 | 2 | 16035 | 2681 |
| FK | 44 | 2 | 5366 | 783 |
| H | 11309 | 593 | 1629380 | 726276 |
| H | 12933 | 603 | 1530603 | 679819 |
| H | 13646 | 631 | 1765965 | 770579 |
| HK | 18 | 2 | 3534 | 618 |
| HK | 19 | 2 | 3883 | 416 |
| HK | 17 | 2 | 2120 | 313 |
| K | 28388 | 1118 | 2992877 | 1619516 |
| K | 23370 | 1169 | 3260261 | 1619510 |
| K | 28174 | 1102 | 2820508 | 1557888 |
| S | 25213 | 731 | 3203800 | 2148850 |
| S | 23520 | 837 | 4304051 | 1811858 |
| S | 31161 | 787 | 4086544 | 1724193 |
| SK | 214 | 11 | 40649 | 10637 |
| SK | 253 | 11 | 36704 | 6743 |
| SK | 228 | 11 | 27629 | 7004 |

FIG. 14. correlations between DIMBOA_hex (upper) and DIMBOA_hex_hex (lower) measured by targeted analysis and untargeted metabolomics (from previous determinations).

### CONCLUSION

No DIMBOA was detected in any of the samples. The concentration of DIMBOA_hex was in the range 4-44291 ng/ml and the concentration of DIMBOA_hex_hex ranged 1-1373 ng/ml. Absolute concentrations by the targeted method were highly correlated with previous untargeted data. The developed method can be used for future targeted analyses of the mentioned compounds.

### REFERENCES

1. WO 98/21978
2. 2. SE9000028-2
3. PCT/SE96/01049
4. WO 98/21978
5. WO 2009/115093
6. WO 97/08202
7. WO 00/38535
8. WO 05/030246
9. De Bruijn, W. J. C. et al. (2016) Mass Spectrometric Characterization of Benzoxazinoid Glycosides from Rhizopus-Elicited Wheat (Triticum aestivum) Seedlings. J. Agric. Food Chem., 64, 6267-6276.
10. Brunius, C. et al. (2016) Large-scale untargeted LC-MS metabolomics data correction using between-batch feature alignment and cluster-based within-batch signal intensity drift correction. Metabolomics, 12, 173.
11. Chambers, M. et al. (2012) A cross-platform toolkit for mass spectrometry and proteomics. Nat. Biotechnol., 30, 918-920.
12. Ganna, A. et al. (2016) Large-scale non-targeted metabolomic profiling in three human population-based studies. Metabolomics, 12, 4.
13. Hanhineva, K. et al. (2011) Qualitative characterization of benzoxazinoid derivatives in whole grain rye and wheat by LC-MS metabolite profiling. J. Agric. Food Chem., 59, 921-927.
14. ELISA Johansson et al., 2009
15. Lönnroth et al., 2015
16. Johansson E, Lonnroth I, Jonson I, Lange S, Jennische E. Development of monoclonal antibodies for detection of Antisecretory Factor activity in human plasma. Journal of immunological methods. 2009; 342(1-2):64-70.
17. Koistinen, V. M. et al. (2018) Metabolic profiling of sourdough fermented wheat and rye bread. Sci. Rep., 8, 1-11.
18. Lin Shi, Johan A Westerhuis, Johan Rosén, Rikard Landberg, C. and Brunius (2018) Variable selection and validation in multivariate modelling. Bioinformatics.
19. Savolainen, O. I. et al. (2016) A Simultaneous Metabolic Profiling and Quantitative Multimetabolite Metabolomic Method for Human Plasma Using Gas-Chromatography Tandem Mass Spectrometry. J. Proteome Res., 15, 259-265.
20. Shi, L. et al. (2018) Plasma metabolites associated with type 2 diabetes in a Swedish population: a case-control study nested in a prospective cohort. 849-861.
21. Smith, C. A. et al. (2006) XCMS: processing mass spectrometry data for metabolite profiling using nonlinear peak alignment, matching, and identification. Anal. Chem., 78, 779-787.
22. Stanstrup, J. et al. (2013) Metabolite profiling and beyond: approaches for the rapid processing and annotation of human blood serum mass spectrometry data. Anal. Bioanal. Chem., 405, 5037-5048.
23. Stekhoven, D. J. and Bühlmann, P. (2012) Missforest-Non-parametric missing value imputation for mixed-type data. Bioinformatics, 28, 112-118.
24. Sumner, L. W. et al. (2007) Proposed minimum reporting standards for chemical analysis. Metabolomics, 3, 211-221.
25. Zhu, Z.-J. et al. (2013) Liquid chromatography quadrupole time-of-flight mass spectrometry characterization of metabolites guided by the METLIN database. Nat. Protoc., 8, 451-460.
26. Torres J, Jennische E, Lange S, Lonnroth I. Clostridium difficile toxin A induces a specific antisecretory factor which protects against intestinal mucosal damage. Gut. 1991; 32(7):791-5.).
27. Lange S. A rat model for an in vivo assay of enterotoxic diarrhea. FEMS Microbiology Letters. 1982; 15(3):239-42.)
28. Bjorck S, Bosaeus I, Ek E, Jennische E, Lonnroth I, Johansson E, et al. Food induced stimulation of the antisecretory factor can improve symptoms in human inflammatory bowel disease: a study of a concept. Gut. 2000; 46(6):824-9.
29. Martos I, Ferreres F, Tomas-Barberan F A. Identification of flavonoid markers for the botanical origin of Eucalyptus honey. Journal of agricultural and food chemistry. 2000; 48(5):1498-502.
30. (Lonnroth I, Oshalim M, Lange S, Johansson E. Interaction of Proteasomes and Complement C3, Assay of Antisecretory Factor in Blood. Journal of immunoassay & immunochemistry. 2016; 37(1):43-54.)

1. A consumable product comprising malted wheat and/or leachate of malted wheat comprising:
(i) 2-methoxyphenol and/or a derivate thereof, and
(ii) 2,4-dihydroxy-7-methoxy-(2H)-1,4-benzoxazin-3(4H)-one-hex and/or 2,4-dihydroxy-7-methoxy-(2H)-1,4-benzoxazin-3(4H)-one-hex-hex,
wherein
a. the concentration of (i) is higher and/or substantially the same as compared to the corresponding non-malted wheat, and
b. the concentration of (ii) is higher as compared to the corresponding non-malted wheat, and
wherein the consumable product induces endogenous production of antisecretory factor (AF) protein and/or fragments thereof in a subject after consumption.

2. A consumable product comprising leachate of malted wheat according to claim 1, wherein said leachate of malted wheat comprises 2,4-dihydroxy-7-methoxy-(2H)-1,4-benzoxazin-3(4H)-one-hex at a concentration of at least 20.000 ng/ml and 2,4-dihydroxy-7-methoxy-(2H)-1,4-benzoxazin-3(4H)-one-hex-hex at a concentration of at least 700 ng/ml, or of at least 2 ng/mg.

3. A consumable product according to claim 1, wherein said consumable product comprises malted wheat and/or leachate of malted wheat in an amount sufficient to induce endogenous production of antisecretory factor (AF) protein and/or fragments thereof in a subject after consumption.

4. A consumable product according to claim 1, wherein said consumable product consists of malted wheat and/or leachate of malted wheat.

5. A consumable product comprising malted wheat and/or leachate of malted wheat according to claim 1, wherein the 2-methoxyphenol derivative is ferulic acid and/or vanillic acid and/or sinapic acid.

6. A consumable product according to claim 1, wherein the malted wheat and/or leachate of malted wheat further comprises:
(iii) a compound selected from the group consisting of 2,4-dihydroxy-(2H)-1,4-benzoxazin-3(4H)-one or a hexose derivative thereof, benzoxazoline-2-one,
and any combination of the foregoing compounds, and
wherein the concentration of (iii) is higher as compared to the corresponding non-malted wheat.

7. A consumable product according to claim 6, wherein the concentration of any one of the compound(s) of (ii) is higher than that of any one of the compound(s) of (iii).

8. A consumable product according to claim 1, wherein the malted wheat is obtained from a process comprising the steps of:
a. wet steeping of wheat at a temperature from about 5° C. to about 20° C.,
b. drying of said wheat,
c. growing of said wheat at a temperature from about 5° C. to about 20° C.,
d. optionally repeating any one of steps a-c, and subsequent
e. drying of said wheat at no more than 80° C.

9. A consumable product according to claim 8, wherein step a, step b, and/or step c independently take place at a temperature of about 8° C., or from about 13° C. to about 15° C.

10. A consumable product according to claim 1, wherein the wheat is selected from the group consisting of Kosack wheat, Festival wheat, Stava wheat, and any combination thereof.

11. A consumable product according to claim 10, wherein the wheat is Festival wheat and/or Stava wheat.

12. A consumable product according to claim 1, which is food, feed, a food supplement and/or a nutraceutical.

13. A consumable product according to claim 1, which has anti-diarrhoeal properties and/or anti-inflammatory properties.

14. A method of treatment, prevention, amelioration and/or prophylaxis of an abnormal physiological condition caused by pathologically high levels of body fluid discharge in a patient in need thereof comprising feeding said patient a sufficient amount of a consumable product according to claim 1.

15. A method of treatment, prevention, amelioration and/or prophylaxis of a condition responsive to increase of levels of antisecretory factor protein and/or antisecretory protein fragments in the blood of a patient comprising feeding said patient a sufficient amount of a consumable product according to claim 1.

16. A method of treatment, prevention, amelioration and/or prophylaxis of a condition according to claim 14, wherein said condition is selected from the group consisting of diarrhoea, inflammatory disease, oedema, autoimmune disease, cancer, tumour, leukaemia, diabetes, diabetes mellitus, glioblastoma, traumatic brain injury, intraocular hypertension, glaucoma, lipid raft dysfunction, compartment syndrome, Alzheimer's disease, Parkinson's disease, encephalitis, and Meniere's disease.

17. A method of treatment, prevention, amelioration and/or prophylaxis of a condition according to claim 15, wherein said condition is selected from the group consisting of diarrhoea, inflammatory disease, oedema, autoimmune disease, cancer, tumour, leukaemia, diabetes, diabetes mellitus, glioblastoma, traumatic brain injury, intraocular hypertension, glaucoma, lipid raft dysfunction, compartment syndrome, Alzheimer's disease, Parkinson's disease, encephalitis, and Meniere's disease.
